(19) United States
(12) Patent Application Publication
(10) Pub. No.: US 2014/0088571 A1
(43) Pub. Date: Mar. 27, 2014

(54) NOVEL DEVICES FOR EFFECTIVE AND UNIFORM SHRINKAGE OF TISSUES AND THEIR UNIQUE METHODS OF USE

(71) Applicant: Trimedyne, Inc., Irvine, CA (US)

(72) Inventor: Marvin P. Loeb, Laguna Woods, CA (US)

(21) Appl. No.: 14/039,090

(22) Filed: Sep. 27, 2013

Related U.S. Application Data
(60) Provisional application No. 61/706,536, filed on Sep. 27, 2012.

(51) Int. Cl. A61B 18/22

(52) U.S. Cl. CPC A61B 18/22
USPC 606/3; 606/16

(57) ABSTRACT

A fiber optical device suitable for treating a wide variety of medical conditions that involve shrinking or tightening of cartilaginous tissue, connective tissue, or muscle tissue comprises an optical fiber capable of laser energy delivery to a predetermined tissue site along with a biocompatible cooling fluid. Illustrative treatable medical conditions are female and male unitary incontinence, female stress urinary incontinence, gastro esophageal reflux disease, obesity, Type 2 diabetes, fecal incontinence, and the like. A preferred laser energy source is a CTH:YAG laser.

### CROSS-REFERENCE TO RELATED APPLICATION

This application claims the benefit of U.S. Provisional Application No. 61/706,536, filed on Sep. 27, 2012, which is incorporated herein by reference in its entirety.

### FIELD OF THE INVENTION

This invention relates to the effective and uniform shrinkage of tissues to treat medical conditions, such as female stress urinary incontinence or “FSUI”, heart valve prolapse, gastro esophageal reflux disease or “GERD”, obesity, Type 2 diabetes, male or female urinary incontinence, male or female fecal incontinence and other medical conditions, which are collectively or individually defined in this Specification and the Claims as “Medical Conditions”. The treatment of such Medical Conditions, which affect millions of people in the United States and up to hundreds of millions in other countries, are costly in lives and a significant cost to the healthcare system.

### BACKGROUND OF THE INVENTION

Many people suffer from Medical Conditions which do not arise from a bacterial or viral infection and have not been conclusively linked to a genetic, hereditary, environmental, dietary or other cause. Some of these Medical Conditions, such as GERD and Type 2 diabetes can be treated with drugs, but the long term use of drugs can cause side effects, and the Medical Condition cannot be completely relieved in many patients. Other Medical Conditions, such as heart valve prolapse, obesity and FSUI can be treated with open heart valve surgery, stomach reduction or “Lap Band” surgery, which can cause adverse effects or death, or the surgical implantation of slings to support the uterus, which can cause infections and other adverse effects, respectively. There is presently no effective therapy for male or female urinary incontinence or male or female fecal incontinence in the elderly.

We accept some of these Medical Conditions as inevitable consequences for some people, and we rely upon surgeries and drugs that are available, despite their cost, adverse effects and the risk of death.

It is an objective of the present invention to safely and effectively apply thermal energy, preferably certain wavelengths of laser energy, to effectively and uniformly shrink certain tissues without damaging adjoining tissues to treat the Medical Conditions described above and others in minimally invasive procedures.

Cartilage, tendons, ligaments, layers of muscle cells and elastic fibers become loose over time, resulting in sphincters not closing properly, causing Medical Conditions such as FSUI, heart valve prolapse, gastro esophageal reflux disease or “GERD”, the premature release of digested or partially digested food from the stomach due to a leaking pyloric valve, causing the loss of the feeling of fullness, overeating and obesity, urinary and fecal incontinence in the elderly and other Medical Conditions.

Weight loss, along with exercise, has been shown to at least partially reduce or reverse Type 2 diabetes, which affects about 60 million people in the U.S. and many millions more outside the U.S.

The cause of loosening of muscle cell and connective tissue layers in sphincters, and the loosening of cartilage, tendons and ligaments is not known, and occurs in some young and middle aged people, so it is not due only to old age.

Cartilage, tendons, ligaments, muscle cell layers and connective tissue layers all contain collagen, a constituent of all soft tissues, to a greater or lessor degree. All of these collagen bearing tissues can be shrunk by thermal cross-linking of collagen. However, if thermal energy is applied to a very small area, like spot welding, the shrinkage effect is not long-lasting. A more uniform and effective means of shrinkage of collagen bearing tissues can be achieved by photo-mechanical cross-linking of collagen, using incoherent or coherent (laser) light. However, continuous emission of incoherent or coherent light energy does not allow any time for the tissue to cool, which can overheat and damage the tissue to be shrunk and adjoining tissues.

CTH:YAG lasers, commonly known as “Holmium” lasers, and fiber-optic laser energy delivery devices used with such lasers, can be used for treating a variety of conditions. One of these conditions is the treatment of herniated spinal discs in minimally invasive, outpatient procedures, by applying Holmium laser energy to vaporize a portion of the excess, benign growth of the nucleus pulposa tissue of a spinal disc, causing the disc to herniated or bulge and press upon nerves in the spinal column; the excess growth of the lobes of the prostate, causing benign prostatic hyperplasia or “BPH”, obstructing urine flow; and the fragmentation of urinary or biliary stones.

Tissues can be shrunk, in proportion to their collagen content, by thermal energy, preferably by photo-mechanical cross-linking of collagen, at temperatures of about 45° to 53° C., preferably at about 48° to 50° C., without affecting their otherwise normal condition or function.

For example, shrinking loose chordae tendinae, which close the leaflets of heart valves, such as the mitral, aortic and tricuspid valves, shrinking muscle cell layers in sphincters which can close at least partially the esophagus, pyloric valve of the stomach, urethra, rectum and anus, as well as shrinkage of the annulus of heart valves, can treat or relieve, for example, such Medical Conditions as heart valve prolapse, GERD, obesity, Type 2 diabetes, and urinary or fecal incontinence. Tissues which can be shrunk to treat a Medical Condition, individually or collectively, are defined in this Specification and the Claims as “Target Tissues”.

Shrinkage by photo-mechanical cross-linking of collagen in tissues is best achieved by certain wavelengths of pulsed laser energy, which allows time between pulses for the tissue to cool and produces a longer lasting effect than thermal cross-linking of collagen by continuously applied radio-frequency (“RF”), ultrasound (“US”) microwave (“MW”) and other forms of thermal energy.

In laboratory testing of application of Holmium laser energy on animal cartilage, we found that cartilage not under tension was shrunk by about 30%. If the cartilage was under tension, the same amount of Holmium laser energy shrank cartilage by about 10-15%.

Of course, if the collagen content is relatively low, or in areas of highly vascularized tissue, with extensive blood flow to carry away the heat, a larger amount of Holmium laser energy or a longer laser energy emission time may be required to obtain such a shrinkage effect.

The use of thermal energy to alter Target Tissues by shrinkage is individually or collectively defined in this Specification and the Claims as to “Alter” or “Altering” a Target Tissue or Tissues.

Altering Target Tissues can be achieved by delivery of various forms of thermal energy, including pulsed laser energy, continuous wave laser energy, pulsed intense incoherent light, continuous wave intense incoherent light, electrically based thermal energy (such as from an electric arc, electrical impedance or resistance), piezo electric (“PE”), electro-shock wave (ESW), radiofrequency (“RF”), microwave (“MW”) or ultrasound (US) energy, the insertion of one or more needles, each containing an optical fiber for delivery of laser energy to a desired depth within a Target Tissue, focusing multiple beams of laser, x-ray, photons, PE, ESW, microwave or ultrasound energy to intersect at a desired point in a Target Tissue (with minimal adverse effect from each individual beam of energy on intervening tissues), a sterile, biocompatible heated liquid (such as water or saline), and other types of thermal energy, are individually or collectively defined to in this Specification and the Claims as “Thermal Energy”.

The Thermal Energies listed above can be delivered to or directed at a Target Tissue, by devices such as optical fibers, lenses, electrodes, wires, cables, tubes, antennae, needles, each containing an optical fiber, straight-firing (0°) optical fiber devices, angled firing (up to 60° from longitudinal axis of the fiber) optical fiber devices and side firing (60° to 90° from longitudinal axis of fiber) optical fiber devices and others.

An electrically or x-ray based source of thermal energy, such as those described above, emit Thermal Energy continuously, not allowing time for the tissue to cool. Also, electrically based Thermal Energy does not produce uniform or complete shrinkage of some Target Tissues, as many electrically based devices tend to follow and dissipate within pathways through tissue with greater salinity (conductivity), such as blood in blood vessels.

Hot gasses or liquids, continuous wave intense light and continuous wave laser energy do not allow time for a Target Tissue to cool and cause thermal damage by heat conduction or diffusion to adjacent tissues. US and MW energy is also usually continuous wave and passes through a Target Tissue to a different extent, based on the density of the tissue, resulting in an erratic effect.

While some continuous wave thermal energy can be gated or pulsed by turning the delivery device “on” and “off”, or periodically interrupting it with an impenetrable barrier, the amount of Thermal Energy delivered is reduced. For example, if the device is “on” for one second and “off” for one second, to allow time for the tissue to cool, the amount of Thermal Energy delivered to a Target Tissue is reduced by 50%.

If the Thermal Energy is “on” for one second and “off” for nineteen seconds, to allow time for the tissue to cool, the amount of Thermal Energy delivered to a Target Tissue is reduced by 95%. Thus, to produce 20 watts of power for one second, with 19 seconds for the tissue to cool, would require a 400 watt laser, which could be costly. Rapidly pulsed RF energy usually raises the temperature of tissue to only about 47° C., rendering it incapable of effectively Altering a Target Tissue, unless very high power RF generators are used, which could be unsafe.

This is not true in the case of certain pulsed lasers, such as Excimer lasers, Chromium, Thulium, Holmium:YAG lasers (often referred to as “CTH:YAG” lasers or simply as “Holmium” lasers), Erbium:YAG lasers, CO₂ lasers and other pulsed lasers, all of which deliver very short, very high peak power pulses of laser energy. Such laser energy, e.g., Excimer, Erbium and CO₂ lasers, require relatively high hydroxyl ion content optical fibers, ultra-low hydroxyl ion content optical fibers, or hollow, silver internally coated optical fibers, respectively, which are expensive. The ability of Excimer, Erbium and CO₂ lasers to efficiently deliver laser energy through such optical fibers is usually limited to about 10 watts.

Also, the light extinction depth of Excimer, Erbium:YAG and CO₂ lasers is very short, only about 5 to 50 microns, and may not reach sufficiently far into a Target Tissue to Alter the Target Tissue. Holmium laser energy, on the other hand, penetrates tissue to a depth of 0.4 millimeters or 400 microns, making Holmium lasers ideal for shrinking Target Tissues to treat various Medical Conditions.

For example, a Holmium laser, producing light energy at a wavelength of 2100 nm, a wavelength of light which is highly absorbed by water, a constituent of all cells, can generate an average of power of up to 100 watts or more of power in pulses of 350 microseconds in duration. At a pulse repetition rate of 10 pulses per second (“Hertz”), a second consists of ten segments of 100,000 microseconds. After each 250 or 350 microsecond pulse of Holmium laser energy, there are 99,750 or 99,650 microseconds for the tissue to cool, until the next laser energy pulse occurs. As a result, coagulation and charring of adjoining tissues is largely avoided, reducing edema and often hastening healing.

Even with thermal diffusion, in a fluid field, consisting of sterile water or saline, at an energy level of about 20 watts over a laser energy emission period of about 30 seconds, with thermal diffusion, Holmium laser energy's aggregate thermal effect on a Target Tissue is only about 1 to 2 mm in depth, depending on the tissue's vascularity.

The short, 0.4 mm tissue penetration depth and short, 250 or 350 microsecond pulses of Holmium lasers provide the ability to precisely, effectively Alter most Target Tissues, with time between pulses for the tissue to cool, without damage to the Target Tissue or adjacent tissues, including blood vessels, ducts and nerves.

Diode, KTP and Nd:YAG lasers, for example, which produce continuous or near-continuous wave energy, penetrate tissue to their light extension depth of about 2 to 4 mm. With thermal diffusion, about 20 watts of laser power of these lasers emitted for about 30 seconds generally penetrates tissue to an aggregate depth of about 5 to 8 mm, several times deeper than Holmium laser energy, and do not allow time for the tissue to cool.

Also, beams of Holmium laser energy diverge from their emission point, Altering a larger area of a Target Tissue than many other types of Thermal Energy, producing a more uniform shrinkage effect.

However, if the Target Tissue is deeper than about 1-2 mm, to avoid thermal damage to intervening tissues, (a) Thermal Energy can be selected based on its light or thermal extinction depth in a particular type, color and density of a Target Tissue, (b) multiple beams of Thermal Energy, converging at a desired depth in a Target Tissue or (c) one or more needles, each containing an optical fiber to transmit laser energy, may be inserted into a Target Tissue to a desired depth to more effectively shrink the Target Tissue.

In a clinical study of the Holmium laser and optical fiber devices, Hanfe et al., Int. J. Med. Sci. 7:120-123 (2010), in the denervation of nerves in the facet joints of the vertebra, 194 patients were treated with a burr to debride or grind-off the outer layer of the capsules of the facet joints, and laser energy was used to coagulate and denervate the exposed nerve endings of the facet joints. At their last visit at three or six years after the therapy, an average of 71% of the patients had at least a 50% reduction of back pain.

By comparison, in the abstract in English of a paper, which was published in German, on a clinical study of 93 patients in which RF energy was used to denervate the nerves of their facet joints, only 50% of the patients had significant pain relief immediately after the RF therapy, only 38% had significant pain relief at 3 months and only 25% of the patients had significant pain reduction at 73 months after the RF therapy (Jerosch et al., Abstract: Z Orthop lhre Grenzgeb, May-June 1993, (3):241-7).

Thermal Energy can be delivered at a desired location in contact with or close to a Target Tissue. aimed in a desired direction and emitted at a desired energy level and for a desired period of time to Alter by shrinking a Target Tissue, after which this process may be repeated at another point or aimed in another direction.

A Thermal Energy delivery device may also be positioned at a desired location in contact with or close to a Target Tissue, aimed in a desired direction and, while Thermal Energy at a desired level and for a desired period of time is emitted, the delivery device may be longitudinally moved back and forth (advanced and withdrawn) at a desired rate of movement over a desired distance for a desired time period, concomitantly or in any desired sequence or order, to apply Thermal Energy longitudinally to the Target Tissue, after which this process may be repeated at another point or aimed in another direction. The above process is defined in this Specification and the Claims as “Moving” or to “Move” a device delivering Thermal Energy.

A Thermal Energy delivery device may also be positioned at a desired location in contact with or close to a Target Tissue, aimed in a desired direction and, while Thermal Energy at a desired energy level and for a desired period of time is emitted, the Source of Thermal Energy may be repetitively rotated laterally over an arc of a desired length, and back to the starting point, at a desired rate of rotation and for a desired time period, to apply the Thermal Energy radially or latitudinally to the Target Tissue, after which this process may be repeated at another point or aimed in another direction. The above process is defined in this Specification and the Claims as “Rotating” or to “Rotate” a Thermal Energy delivery device.

Also, a Thermal Energy delivery device can be positioned at a desired location, in contact with or close to a Target Tissue, aimed in a desired direction and, while Thermal Energy at a desired energy level and for a desired period of time is emitted, the delivery device may be Moved and Rotated, concomitantly or in any desired sequence or order, to Alter a Target Tissue by shrinkage, after which this process may then be repeated at another point or aimed in another direction. The above process of Stationing, Moving and Rotating a Source of Thermal Energy is defined in this Specification and the Claims as “Sweeping” or to “Sweep” a of Thermal Energy delivery device.

Any or all of the above processes of Stationing, Moving, Rotating and Sweeping any of the Thermal Energy delivery devices can be separately employed, concomitantly applied, or employed in any desired order or sequence.

The Thermal Energy delivery device, the direction in which it is aimed, the time period of Thermal Energy emission, the distance and rate of movement, the time period thereof, the length of each arc, the rate of rotation and the time period thereof, in Stationing, Moving, Rotating and/or Sweeping a Thermal Energy delivery device are based upon (a) the type, density, color, thermal absorption coefficient and volume of the Target Tissue to be Altered, by shrinkage and (b) the environment or field in which the process is performed, whether in an aqueous field, which cools the Target Tissue, in a air or CO₂ gas field with or without a spray of sterile water or saline, in a cooled gas environment, preferably a cryogenically cooled gas, to cool the Target Tissue. Such environments are individually or collectively defined in this Specification and the Claims as the “Environment” in which the Altering by shrinkage of a Target Tissue occurs.

If the Environment does not include the infusion of a sterile, biocompatible irrigating fluid or a spray of such fluid to cool the Target Tissue, a much lower level of Thermal Energy is used to avoid charring and damage to the Target Tissue and adjacent tissues, as is described hereinbelow.

Also, in this Specification and the Claims, the following terms: (a) to treat, delay progression of or prevent a Medical Condition are individually or collectively defined herein as “Treating” or to “Treat” a Medical Condition; (b) a person suffering from a Medical Condition is defined herein as a “Patient”; and (c) delivering Thermal Energy at, onto the surface of or into a Target Tissue is individually or collectively defined herein as “Onto” said Target Tissue.

A variety of Medical Conditions can be Treated by Stationing, Moving, Rotating and/or Sweeping a Source of Thermal Energy Onto a Target Tissue, such methods of delivering Thermal Energy include the following:

A method for Treating a Medical Condition of a Patient comprised of at least one of: Stationing, Moving, Rotating and Sweeping a Source of one of: pulsed laser energy and continuous wave laser energy, delivered Onto a Target Tissue at an angle of one of: 0°, up to 60° and 60° to 90° from the longitudinal axis of the optical fiber in the Thermal Energy delivery device, one or more needles, each containing an optical fiber for delivery of laser energy to a desired depth within a Target Tissue, and multiple beams of at least one of: laser, x-ray, proton, RF, MW, U.S. and ESW energy, focused to intersect at a desired point, to shrink at least one of: (a) the chordae tendinae of a heart valve, (b) the annulus of a heart valve, (c) the sphincter of the esophagus, (d) the sphincter of the pyloric valve of the stomach, (e) the sphincter of the male or female urethra, (f) the sphincter of the male or female rectum, (g) the sphincter of the male or female anus, and (h) the tendons and ligaments that hold the uterus in place, the Medical Condition being at least one of: (i) heart valve prolapse, (ii) GERD, (iii) obesity, (iv) Type 2 diabetes, (v) male or female urinary incontinence, (vi) male or female fecal incontinence and (vii) FSUI, as well as other Medical Conditions which can be Treated by shrinking a Target Tissue.

For example, to Alter by shrinkage a Target Tissue, the Thermal Energy may be oriented to emit laser energy at one of: (a) Onto the Target Tissue or (b) aimed to emit Thermal Energy at about 3 o'clock (where 12 o'clock is at the top surface of the Target Tissue) and, if desired, the Thermal Energy beam may be Rotated through an arc of about 90° (from about 1:30 to 4:30 o'clock) to 120° (from about 1 to 5 o'clock), while Thermal Energy is emitted at a desired power level for a desired period of time, after which the thermal energy beam is successively aimed to emit at 6, 9 and 12 o'clock and the above described 90° to 120°. Rotating process is repeated at each such o'clock position.

If desired, the of Thermal Energy delivery device may be successively Moved, before, during, or after each Rotating process, to another of a series of locations, at which the Rotating process can be repeated. Or, if desired, the Thermal Energy deliver device may be successively Moved, Rotated and/or Swept between a series of two or more locations, in any desired sequence, individually or in any combination of the above processes.

A Thermal Energy delivery device may be introduced into the body by one of: (a) a body orifice, (b) an open surgical procedure, (c) a surgically created passageway, (d) an endoscopic procedure, (e) a laparoscopic procedure and in other ways.

The term “Rotated” as used herein, means repetitive rotations of the Source of Thermal Energy from its starting point to its end point and back, during the selected rotation time period, such as 0.5 to 2 cycles per second, preferably about one cycle per second, so the operator can time each arc by his or her mentally counting “one thousand”, “two thousand”, etc.

Target Tissues can be Altered by Stationing, Moving, Rotating and/or Sweeping a Thermal Energy delivery device, in any desired sequence or combination, Onto a Target Tissue. One of the preferred types of Thermal Energy is laser energy, preferably pulsed laser energy, most preferably pulsed CTH:YAG or Holmium laser energy, which may be transmitted through a straight-ahead firing, an up to 60° angled firing, or a 60° to 90° side firing laser energy delivery device, as described below.

In the first side firing embodiment of a laser energy delivery device of the present invention, the proximal end of a conventional, end-firing optical fiber is optically coupled to a source of laser energy and a metal tip for diverting laser energy laterally from the axis of the optical fiber is fixedly attached by crimping and/or an adhesive to the distal end of the optical fiber. The metal tip is preferably made entirely of or coated with a material highly reflective to the wavelength of laser energy being used, such as silver or gold, stainless steel which has been plated with silver or gold, with a thickness of preferably at least five or more thousandths of an inch, stainless steel with an insert of gold or silver, preferably with a thickness of ten to twenty or more thousandths of an inch, or stainless steel coated with a dielectric.

Preferably, the protective buffer coating and any polymer cladding are removed from the distal end portion of the optical fiber, prior to attachment of the metal tip. Alternatively, the metal tip can also be attached by an adhesive and/or crimping to the protective buffer coating covering the optical fiber, if desired.

The metal tip defines a central cavity, into which the distal end of the optical fiber extends. The distal end surface of the cavity is inclined at an angle of about 35° to 50°, preferably at an angle of about 45°. The open portion of the cavity allows laser energy, reflected by the inclined, reflective metal surface, to be emitted from the cavity in the metal tip at an angle of about 90° from the axis of the optical fiber, in accordance with Snell's Law.

For ease of manufacture and durability, the entire metal tip is preferably made of a highly reflective material, such as gold or silver having a purity of at least about 90%, both of which are easily malleable, preferably silver, which has about the same reflectivity as gold, but is much less expensive. Most preferably, the gold and silver are at least about 95.5% pure.

In the side firing device described above, the optical fiber extends from the Source of laser energy, through a passageway or channel, which extends lengthwise through a metal or rigid plastic handpiece, for ease of use.

The optical fiber extends through the passageway and is fixedly attached within the proximal end of the handpiece by an adhesive or the like, which serves to sealingly close the proximal end of the passageway in the handpiece. Alternatively the optical fiber may be removably attached within the proximal end of the handpiece by a compression fitting, as known in the art, which sealingly closes the proximal end of the handpiece.

In addition to sealingly closing the distal end of the handpiece, the compression fitting, when loosened, enables the side firing device to be removed, cleaned and resterilized for use in another procedure, and enables the handpiece to be cleaned, resterilized and used again or vice versa. The optical fiber extends distally from the handpiece a desired distance, with its distal end modified to emit laser energy laterally from the axis of the optical fiber, as described above.

Optionally, the optical fiber of the side firing device can extend through a plastic cannula extruded with a central or eccentric channel for the optical fiber and one or more surrounding channels for other purposes. The plastic cannula can be made of a flexible, semi-flexible, semi-rigid or rigid biocompatible plastic, preferably a very flexible plastic.

The proximal end of the plastic cannula may be fixedly attached by an adhesive or other means within (a) the distal end of the connector of the optical fiber at or near the laser, (b) preferably at least about 6 cm proximal from the proximal end of the handpiece or (c) within the distal end of the handpiece.

The distal end of a multi-channel cannula can extend (a) up to the proximal end of the crimped portion of a metal tip, (b) over the crimped portion of a metal tip, (c) over the crimped portion and over a metal tip, up to the area of laser energy emission or (d) over the crimped portion and up to the distal end of a metal tip, with a port for emission of laser energy over a 45° inclined surface of a metal tip. For example, the optional, multi-channel plastic cannula can be extruded with a central channel for the optical fiber to center the side firing device within a blood vessel, bronchi, hollow organ or duct, or with an eccentric channel for the optical fiber to position the side firing device close to the wall of the blood vessel, bronchi, hollow organ or duct.

The central or eccentric channel for the optical fiber can have, for example, three surrounding longitudinal channels, one channel for infusion of a biocompatible irrigation fluid, such as sterile saline or water, to clean and cool the laser energy emitting surface of the side firing device and the Target Tissue, one channel for infusion of a biocompatible fluid, such as sterile, saline or water, to inflate a concentric, eccentric or back mounted balloon, which may be attached to the exterior of the multi-channel cannula proximal to the proximal end of the metal tip, to either (a) center the side firing device, for example, in the annulus of a prolapsed heart valve, the sphincter of the esophagus, the sphincter of the pyloric valve of the stomach, the sphincter of the male or female urethra, the sphincter of the male or female rectum or the male or female anus, or (b) to position the laser energy emitting surface of the side firing device close to at least one of the above, with one channel to enable a sterile, biocompatible fluid to inflate the balloon and one channel to enable such fluid to be withdrawn from and deflate the balloon.

If a plastic cannula is extruded with two surrounding channels and extends from the handpiece to a point just proximal to the laser energy emission port of the side firing device, one channel can be used for infusion of a sterile biocompatible irrigation fluid to cool and flush debris from the optical components at the distal end of the side firing surface of the device and cool the Target Tissue, and one channel can be used to inflate and deflate a balloon mounted on the side firing device, as described above.

In the cannula described above with only two surrounding channels, the balloon can be manufactured with one or more holes or vents to allow the air to escape into the atmosphere when one channel in the cannula is used to inflate the balloon with a fluid.

If the cannula is extruded with three channels, one channel can be used to cool and clean the optical components at the distal end of a side firing device and cool the Target Tissue, one channel can be used to inflate the balloon and one channel can be used to deflate the balloon.

Of course, any number of channels can be used to achieve their respective, desired functions.

As mentioned above, the balloon can be concentric to center the side firing device in a body orifice, blood vessel, duct, hollow organ or surgically created passageway; eccentric, preferably wider on the side opposite the side from which laser energy emitted, and narrower on the side from which laser energy is emitted, to position the laser energy emitting side of the side firing device close to the Target Tissue; or the balloon may be mounted on the side of the side firing device opposite the side from which laser energy is emitted, to force the side firing device against the Target Tissue.

A luer fitting may be sealingly and fixedly attached within and extends through the body of the handpiece and is in fluid communication with the central passageway in the handpiece and the irrigation fluid channel of the multi-channel cannula. To inflate a balloon, a separate luer fitting can be attached to the plastic cannula in fluid communication with the channel in the plastic cannula for inflation of the balloon, and a third luer fitting can be attached to the cannula in fluid communication with the fluid return channel, to enable the returned fluid to flow through a tube, for example, into a syringe, collection bottle or drain.

Alternatively, all luer fittings can be attached to the multi-channel cannula, each in fluid communication with one channel of the multi-channel cannula, at one point or points at least about 6 cm proximal to the proximal end of the handpiece, so the luer fittings and the attached fluid lines do not interfere with the surgeon's use of the handpiece to Station, Move, Rotate and Sweep the distal, side firing portion of the device.

To provide support for the luer fittings at their junction with each of their respective channels of the hollow, multi-channel cannula, a rigid plastic or metal collar can be adhesively attached to the multi-channel cannula and the luer fitting fluid lines. All three luer fittings may be attached with a radial collar, a collar extending longitudinally along the exterior of the multi-channel cannula, or a separate collar for each luer fitting may be employed.

If the multi-channel cannula extends over the laser energy emitting portion of the side firing device, the multi-lumen cannula must have a port for emission of laser energy opposite the surface from which laser energy is emitted.

In the second embodiment of a laser energy delivery device embodying the present invention, an optical fiber, optically coupled to a source of laser energy, from whose distal end portion the plastic buffer coating and any polymer cladding has been removed, a process called “baring” the optical fiber or producing an optical fiber with a “bared” distal end portion. The optical fiber extends through a hollow passageway extending lengthwise through the body of a handpiece. The optical fiber is fixedly attached to the handpiece, preferably within the proximal end of the handpiece, in a manner which sealingly closes the proximal end of the passageway, or allows the optical fiber to be sealingly and removeably attached in the proximal end of the handpiece, as described above.

After baring the distal end portion of the optical fiber, the distal end of the optical fiber is beveled at an angle of about 35° to 45°, preferably at an angle of about 38° to 44°, and most preferably at an angle of about 40° to 41° for optimal reflection and laser energy transmission efficiency. A distally closed-ended capillary tube is disposed over and fixedly and sealingly attached by an adhesive, thermal fusing, a combination of the foregoing or other means known in the art, to the bared distal end portion of the optical fiber. Fixedly and sealingly disposing a closed-ended capillary tube over the distal end of the optical fiber creates an air environment opposite the beveled, distal end surface of the optical fiber.

The difference in the refractive index of air, versus the refractive index of the quartz or fused silica core of the optical fiber, enables total internal reflection of the light energy to occur laterally at an angle of double the bevel angle, according to Snell's Law. If the distal end of the optical fiber is beveled at an angle of 40° to 41°, laser energy is emitted at an angle of about 80° to 82° out of a side laser energy emission port.

Likewise, the optional plastic cannula described above, the optional balloon configurations described above, the use of the channels described above and the luer fittings communicating with each of the surrounding channels of the multi-channel cannula, as described above, can be used with this second embodiment of the side firing device. Alternatively, the plastic cannula can extend over the side firing device, with a port for emission of laser energy positioned in the path of laser energy emission, as described above.

However, in the third embodiment of the device of the present invention, if laser energy at wavelengths of about 1400 to 1500 nanometers (nm) or 1800 to 3000 mm, which wavelengths are highly absorbed by water, is emitted through an optical fiber, whose distal end has been beveled at an angle of 35° to 45°, preferably at an angle of about 40° to 41° for optimal reflection and laser energy transmission efficiency, in an aqueous liquid environment, we have found that the closed-ended capillary tube can be eliminated for some applications. The first portion of the laser energy emitted vaporizes a portion of the aqueous irrigation liquid infused through an endoscope, or a channel in the optional multi-channel plastic cannula described above, and creates a steam bubble to form opposite the beveled, distal end surface of the optical fiber.

The steam bubble has an index of refraction sufficiently lower than that of the refractive index of the quartz or fused silica core of the optical fiber to cause the laser energy to be internally reflected, according to Snell's Law, at an angle of about 80° to 82° out of the side port in the liposuction cannula, as described above. However, the laser energy emitting surface of this embodiment of the present invention must be positioned close to but not in contact with the Target Tissue, or much of the laser energy will be wasted vaporizing any intervening aqueous irrigation liquid. Contacting the Target Tissue can cause tissue to adhere to the laser energy emitting surface of the side firing device, reducing its transmission efficiency.

Likewise, the optional multi-channel cannula described above, the optional balloon configurations described above, the surrounding channels described above, optionally extending the multi-channel cannula over the side firing device, with a port for emission of laser energy, as described above, and the luer fittings in fluid communication with each of the surrounding channels of the cannula, can be used with this embodiment of the side firing device.

For use in surgically created passageways, or in endoscopic or laparoscopic procedures, an aiming beam of a desired color, for example, red or green, such as from a helium neon (HeNe), a diode laser or other laser emitting about 1 to 5 milliwatts of power, as known in the art, can be transmitted through the optical fiber and reflected at about the same angle as the therapeutic laser energy, which may be of an invisible wavelength, to enable the operator to see the direction in which the therapeutic laser energy is being emitted. Green is preferred, as red may be more difficult to discern in an area containing blood.

Laser energy at wavelengths of about 300 to 400 nm are used through optical fibers with a relatively high hydroxyl ion content of 600 to 800 ppm, called high-OH fibers to prevent excessive loss of laser energy at these wavelengths. Laser energy at wavelengths of about 400 to 1400 nm and about 1500 to 1800 nm can be used through conventional optical fibers with a hydroxyl ion content of 100 to 600 ppm or, preferably, for more efficient transmission efficiency, through or optical fibers with a low hydroxyl ion content, of about 0.1 to 100 ppm; to reduce transmission losses.

An optical fiber with a low hydroxyl ion (water) content of less than about 100 parts per million, preferably about 1 to 100 parts per million (“ppm”), called a low-OH fiber, is used with lasers whose wavelength is 1400 to 1500 or 1800 to 2300 nm, to prevent excessive loss of laser energy. And, an optical fiber with an extremely low hydroxyl ion content of about 0.01 to 1 ppm, called an ultra low-OH fiber, is used with lasers emitting energy at a wavelength of 2300 to 3000 nm, to avoid excessive loss of laser energy at these wavelengths.

Contrary to common wisdom in the laser field, we discovered that all wavelengths of laser energy from about 300 nm to 3000 nm, used through optical fibers with hydroxyl ion contents applicable to each, as described above, can be effectively used in the side firing device described above, in which the distal end of the optical fiber is beveled at an angle of about 35° to 45°, most preferably at an angle of about 40° to 41°, and is fixedly and sealingly encased by a distally closed-ended capillary tube to create the air environment, which is required for total internal reflection of laser energy to occur.

A variety of lasers fall within wavelengths of about 300 nm to 3000 nm. For example, lasers emitting at 300 to 400 nm, include, for example, excited dimer lasers, called “eximer” lasers, including Xenon Chloride (XeCl) lasers emitting at a wavelength of about 308 nm and Xenon Fluoride (XeFl) lasers emitting at a wavelength of about 351 nm, which wavelengths are highly absorbed by molecular bonds, causing disruption and vaporization of tissue. However, the light extinction depth of excimer laser energy is only about 5 microns, excimer lasers are generally limited to powers of only about 10 watts, and they use highly toxic gasses, which can be dangerous in a medical facility.

Lasers emitting at 400 nm to 1400 nm and from 1500 nm to 1800 nm include, for example, an argon laser emitting at about 488 to 514 nm, a KTP laser emitting at a wavelength of 532 nm, which is highly absorbed by a red pigment, such as oxygenated hemoglobin in blood, a diode laser emitting at wavelengths of about 600 nm to 1400 nm, an alexandrite laser emitting at a wavelength of 810 nm, and a Nd:YAG laser emitting at a wavelength of 1064 nm, which wavelengths are absorbed to a modest extent by pigments and to a limited extent in water. These lasers have light extinction depths ranging from 800 to 4000 microns.

Lasers emitting at 1400 to 1500 nm and from 1800 to 3000 nm include, for example, a certain diode laser emitting at a wavelength of about 1470 nm, a Thulium:YAG laser emitting pulsed or continuous wave laser energy at a wavelength of about 2000 nm, a Chromium, Thulium, Holmium or CTH:YAG laser, commonly referred to as a “Holmium laser”, emitting pulsed laser energy at a wavelength of about 2100 nm, a YSGG:YAG laser emitting pulsed laser energy at a wavelength of about 2106 nm, the light extinction depth of the CTH:YAG and YSGG:YAG lasers in tissue is about 400 microns, and an Erbium:YAG laser emitting pulsed laser energy at a wavelength of about 2900 nm, whose light extinction depth in tissue is only about 50 microns, all of which wavelengths are highly absorbed by water, a constituent of all tissues, as well as the irrigation liquids commonly used in endoscopic procedures.

While all of the above-described wavelengths of laser energy can be used in the side firing device of the present invention, provided the core of the optical fiber has a sufficiently low hydroxyl-ion content of an appropriate amount for effective transmission of each laser's wavelength, pulsed Holmium laser energy is preferred, as its depth of penetration in tissue is ideal for use in arteries, veins, bronchi and other Target Tissues with a wall thickness of about 1 to 2 mm. And, it's very short, about 350 microsecond pulses of laser energy, leave time for the tissue to cool between pulses of laser energy.

If the wall thickness of a Target Tissue is larger than 1 to 2 mm, (a) a longer emission time may be used, to enable thermal diffusion of the laser energy to occur, (b) a laser whose wavelength penetrates tissue to a greater depth can be employed, (c) multiple beams of laser energy or other Thermal Energy may converge at a desired point within the Target Tissue, or (d) one or more needles, each containing an optical fiber, may be inserted to deliver laser energy at a desired depth within the Target Tissue.

The handpiece can have a raised button, whose color may be significantly different from that of the handpiece, which the operator can see and sense by tactile feel, as known in the art. The button can be positioned on the side of the handpiece from which the laser energy is emitted or, preferably, on the side of the handpiece opposite the side from which the laser energy is emitted. If so positioned, when the handpiece is gripped, the forefinger or thumb of the operator, touching the button, points in the direction in which the laser energy will be emitted, as known in the art.

In a preferred version of the second embodiment of the present invention, we discovered that the beveled, distal end surface of the optical fiber may be encased within a closed-ended capillary tube with a substantially thinner wall thickness, which causes the laser energy to be more widely diverged, enabling a greater volume of Target Tissue to be Altered and allows the side firing device to be rotated through an arc of only about 90° to achieve the same effect. In this embodiment, the wall thickness of the capillary tube is not greater than 350 microns, compared to a typical wall thickness of about 500 to 600 microns of the capillary tube in the second embodiment of the side firing devices described above.

Today, all side firing devices, of which we are aware, are made with optical fibers with a core diameter of about 500 to 600 microns, as conventional wisdom in the medical laser field is such core diameters are necessary to efficiently capture and transmit 100 or more watts.

Contrary to common wisdom, we have tested successfully optical fibers with core diameters less than 500 microns and discovered that 100 watts of Holmium and other wavelengths of laser power can be efficiently transmitted through optical fibers as small as 365 microns or even smaller. In the process of constructing and testing optical fibers with a core diameter of 365 microns, we created the smallest side firing devices ever made, with an O.D. of no more than 1.5 mm (conventional side firing devices with an internal fluid channel are usually 2.0 mm to 2.5 mm in O.D.

These smaller diameter core fibers enable side firing devices to be used through a metal cannula with a bend near its distal end, a conventional guiding catheter or a rigid endoscope, whose distal end may be flexible and bent or articulated by wires or other means, by up to 90° or more, provided the bend radius is not smaller than 1 to 1.5 cm, as described below, which could cause laser energy to leak at the bend and damage the cannula, guiding catheter or flexible endoscope.

Another improvement we conceived is the use of a thin, heat shrinkable plastic tube, which is shrunk over the distal end portion of the optical fiber, the junction of the optical fiber with the proximal end of the metal tip or the proximal end portion of the capillary tube and terminates just proximal to the area of laser energy emission from the metal tip or capillary tube. The heat shrunk tube reduces the risk of the capillary tube of the second embodiment of the present invention from being dislodged from the optical fiber, as a safety measure. An adhesive may also be applied to the area to be covered by the heat shrinkable tube, prior to the heat shrinking process, as an additional safety measure.

The unique construction of any of the side firing devices described above may be employed to effectively and uniformly Alter a Target Tissue by shrinkage. These side firing devices can be used in one or more novel methods of use to achieve a significantly more effective, safe and uniform Altering by shrinkage of a Target Tissue to Treat a Medical Condition of a Patient, as described below.

After Stationing a side firing device or other Source of Thermal Energy opposite a Target Tissue, the button on the handpiece can be positioned, for example, at 3 o'clock, causing Thermal Energy to be emitted at 9 o'clock, after which the button can be successively positioned at 6, 9 and 12 o'clock, causing Thermal Energy to be emitted at 12, 3 and 6 o'clock. This process can be started at any of such positions.

Thermal Energy can be emitted, for example, at a power level of about 3 to 40 watts, preferably about 8 to 20 watts, provided the emission of Thermal Energy is in an aqueous Environment or is concomitantly accompanied by the infusion of a sterile, biocompatible irrigating fluid or a spray of a sterile, biocompatible irrigation fluid, preferably sterile water or saline or a cold or cryogenically cooled biocompatible gas, such as CO₂ or nitrogen, to cool the Target Tissue.

However, if the Target Tissue is not concomitantly cooled, lower laser power must be applied, for example, at about 0.05 to 10 watts, preferably at about 0.1 to 3 watts, to avoid a build-up of thermal energy that could damage the Target Tissue or adjacent tissue. The level of laser energy and its duration is dependent on the area and volume of Target Tissue to be Altered, and the rate of Moving, Rotating and/or Sweeping the laser energy delivery device and the time period thereof, and is determined by the physician performing the procedure.

The button on the handpiece can be positioned, for example, at 12 o'clock, and laser energy or other Thermal Energy may be emitted for about 5 to 30 seconds, preferably for about 10 to 20 seconds, at 6 o'clock, while repetitively Rotating the cannula back and forth through an arc of about 90° to 120°, from up to about 4 to 8 o'clock, as the side firing device or other Thermal Energy delivery device is positioned or Moved. Then, the button can be successfully positioned at 3, 6 and 9 o'clock and the above described process of delivering the Thermal Energy can be repeated.

If the side firing device or other Thermal Energy delivery device is Moved, each longitudinal movement can be for about 1 to 5 seconds, preferably about 2 to 3 seconds, in each direction, depending upon the distance the side firing device is to be extended from the distal end of a cannula, endoscope, laparoscope or though a surgically created passageway, as determined by the physician performing the procedure.

If the side firing device or other Thermal Energy delivery device is Rotated through an arc of about 90° to 120° while positioned and/or Moved, its rate of rotation is preferably about one arc per second, for the reasons described above.

To Treat certain Medical Conditions of a Patient, it may be difficult, impossible or impractical to use a side firing device. In such instances, a prior art optical fiber may be inserted through a rigid endoscope or laparoscope, or through a rigid endoscope or laparoscope whose distal end portion, about 5 to 10 cm in length, may be articulated or bent at an angle up to at least 90°, usually by manipulating wires contained in the distally flexible device, as known in the art, provided the bend radius is not smaller than 1 to 1.5 cm, as described below. The above described scopes can be positioned, Moved, Rotated and/or Swept, individually or in any desired combination or sequence, to Alter the Target Tissue to Treat the Medical Condition.

Also, in the treatment of certain Medical Conditions, an optical fiber may extend from a source of laser energy through a handpiece and a prior art, hollow metal or rigid plastic cannula, preferably made of medical grade stainless steel. The proximal end of the optical fiber is fixedly attached within the distal end of the handpiece by an adhesive, as known in the art.

The prior art cannula's distal end portion can be straight (0°) or bent at an angle of, for example, 10°, 20°, 30°, 40°, 50° or 60°, or any other desired angle, providing the bend radius does not exceed 1 to 1.5 cm, as described below. Such a cannula and the optical fiber extending therethrough, may be inserted through a body orifice or a surgically created passageway and used under direct viewing, (a) through a rigid endoscope or laparoscope, (b) through an endoscope or laparoscope with a distal, flexible portion, or (c) guided by ultrasound, fluoroscopic or x-ray imaging. The optical fiber device can be Stationed, Moved, Rotated and/or Swept, in any desired sequence, individually or in any desired combination, to Alter a Target Tissue to Treat a Medical Condition of a Patient.

However, if a Thermal Energy delivery device is used in an air or biocompatible gas environment, without the infusion or a spray of a sterile, biocompatible irrigation fluid, it must be used at the low energy levels described above to avoid excessive thermal damage to the Target Tissue or adjoining tissues.

If used in an air or biocompatible gas environment, we found that the prior art, cannula/optical fiber device described above can contain a space between the exterior of the optical fiber and the inner surface of the cannula for infusion of a sterile, biocompatible fluid, such as saline, water, a cooled gas, preferably a cryogenically cooled gas, such as CO₂ or Nitrogen, to cool the optical fiber and the Target Tissue.

A luer fitting, in fluid communication with the space between the exterior of the optical fiber and the interior of the cannula, as known in the art, may be attached to the handpiece or the cannula, as described above, can be used to infuse the cooling fluid. Cooling the optical fiber and the Target Tissue enables a substantially greater level of laser energy to be safely used.

Other variations of the above described devices can be made and other Sources of Thermal Energy to Alter a Target Tissue can be used to Treat a variety of Medical Conditions of Patients, without departing from the principles set forth herein and without limiting the intent and scope of the present invention.

### BRIEF DESCRIPTION OF THE DRAWINGS

FIG. 1 is an external, side view of the first embodiment of the device of the present invention, with an expanded, partial, cross-sectional, side view of the distal end portion of the device.

FIG. 2 is a partial, cross-sectional, side view of the distal end portion of the second embodiment of the device of the present invention.

FIG. 3 is a partial, cross-sectional, side view of the distal end portion of the third embodiment of the device of the present invention.

FIG. 4 is a partial, cross-sectional, side view of the distal end portion of an improved embodiment of the device of FIG. 2.

FIG. 5 illustrates the laser energy emission area resulting from positioning and Rotating the embodiment of the device of FIG. 2.

FIG. 6 is a partial, cross-sectional, side view of the distal end portion of a further improved embodiment of the device of FIG. 2.

FIG. 7 is a partial, cross-sectional, side view of another improved embodiment of the device of FIG. 6.

FIG. 8 is a cross-sectional, end view at plane A-A of the device of FIG. 7.

FIG. 9 is a partial, cross-sectional side view of another improved embodiment of the device of FIG. 2.

FIG. 10 is a cross-sectional, end view at plane B-B of the device of FIG. 9.

FIG. 11 is a partial, cross-sectional, side view of another improved embodiment of the device.

FIG. 12 is a cross-sectional, end view at plane C-C of the device of FIG. 11.

FIG. 13 is a partial, cross-sectional, side view of another improved embodiment of the device.

FIG. 14 is a partial, cross-sectional, top view of the handpiece and luer fittings of the present devices.

FIG. 15 is a schematic representation of four methods of use of the device of the present invention.

FIG. 16 is a partial, external, side view of four prior art optical fiber/cannula devices.

FIG. 17 is a partial, external, side view of four improved optical fiber/cannula embodiments of the device of the present invention.

FIG. 18 is a cut-through, top view of the female reproductive system.

FIG. 19 is a cut-through, side view of a sectioned heart.

FIG. 20 is a partially cut-through, side view of the esophagus, stomach and duodenum.

FIG. 21(a) is a cut-through, side view of the male penis and urethra.

FIG. 21(b) is a cut-through, side view of the female bladder and urethra.

FIG. 22 is a cut-through, side view of the rectum and anus.

### DETAILED DESCRIPTION OF THE INVENTION

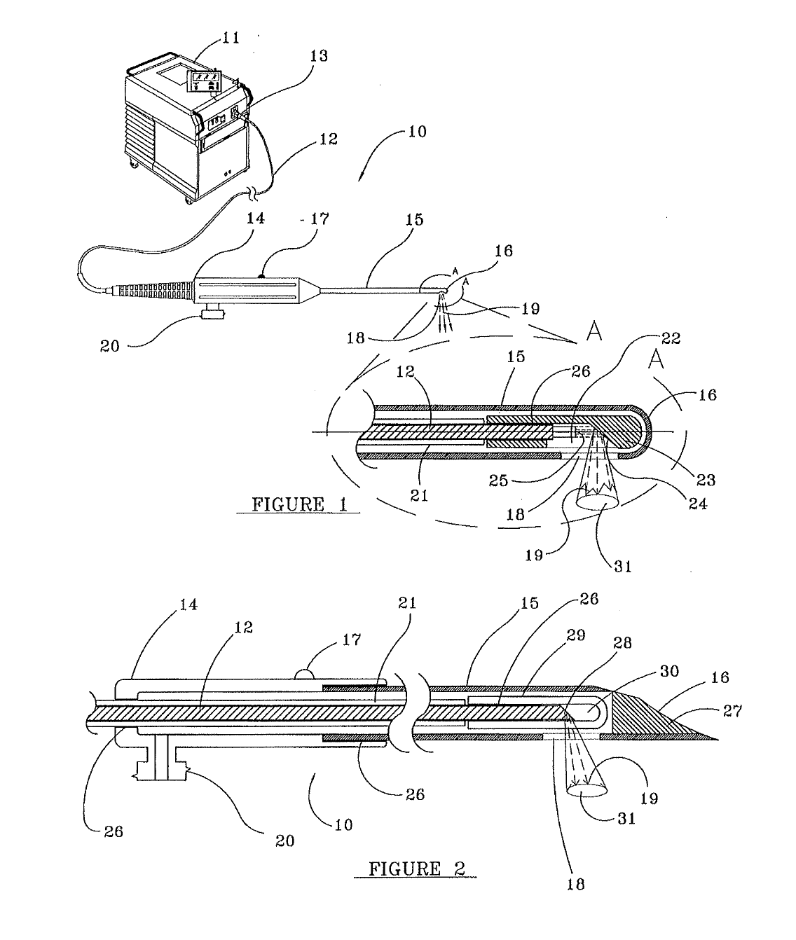

The first embodiment of side firing device 10 suitable for practicing the present invention is illustrated in FIG. 1. Device 10 is comprised of laser energy source 11 and optical fiber 12. Connector 13 operably couples optical fiber 12 to laser energy source 11. Optical fiber 12 is fixedly and sealingly attached within the proximal end of handpiece 14 by adhesive 26, as known in the art, and extends through a hollow, longitudinal, passageway (not separately shown) in handpiece 14 and is in fluid communication with hollow metal or rigid plastic cannula 15, preferably of medical grade stainless steel, whose proximal end is fixedly attached by adhesive 26 within the distal end of handpiece 14.

The distal end 16 of cannula 15, as shown in FIG. 1, is rounded. Distal end 16 of cannula 15 may also be blunt, sharp, double-bevel needle-shaped, trocar shaped or of any other desired shape, as known in the art. Using a needle-like or sharp-ended cannula within a patient entails considerable risk to the patient, should be used under endoscopic, ultrasound or x-ray imaging and requires greater care by the surgeon.

Alternatively, optical fiber 12 may be removeably and sealingly attached within the proximal end of handpiece 14 by a compression fitting (not separately shown), as known in the art, enabling side firing device 10 to be removed, cleaned, sterilized and reused, if desired.

Button 17 on handpiece 14, in this embodiment, is preferably positioned on the side of handpiece 14 opposite the side of handpiece 14 from which the emission of laser energy occurs through laser energy emission port 18 in cannula 15, as shown by arrows 19, resulting in laser energy spot area 31 on or within a Target Tissue. While button 17 may also be positioned on the side of handpiece 14 from which the emission of laser energy occurs, button 17 will be less able to be visualized during use.

Luer or other fluid connector fitting 20, which is fixedly attached within and extends through the wall of handpiece 14, is in fluid communication with the longitudinal passageway (not separately shown) in handpiece 14, hollow cannula 15 and port 18 positioned over the source of emission of laser energy. Luer fitting 20 enables a sterile, biocompatible fluid, such as saline or water, to be infused through longitudinal passageway (not separately shown) in handpiece 14 into hollow cannula 15, to clean and cool the laser energy emitting surface of side firing device 10 and cool the Target Tissue.

As shown in the cut-through, expanded view A-A of the distal end portion of device 10, buffer coating 21 and any optional polymer cladding (not separately shown) of optical fiber 12 have been removed from the distal end portion of optical fiber 12, which extends into cavity 22 in hollow metal end piece 23. Metal end piece 23 is fixedly attached to the bared distal end portion of optical fiber 12 by adhesive 26, crimping of the proximal end portion of metal end piece 23 to optical fiber 12 (not separately shown) or both, or by other means known in the art.

As illustrated in expanded view A-A, metal end piece 23 and optical fiber 12 are disposed within metal or plastic hollow cannula 15, whose distal end 16 may be rounded, as shown, sharp, conical, blunt or of any other desired shape. Cavity 22 in metal end piece 23 is formed with a reflective, inclined surface 24 opposite distal end face 25 of optical fiber 12. Reflective surface 24 of metal end piece 23 is inclined at an angle of about 35° to 50°, preferably about 45°, to reflect the laser energy from inclined reflective surface 24 at an angle of about 90° from the axis of optical fiber 12, according to Snell's law, out of port 18, as shown by arrows 19.

Metal end piece 23 can be made entirely of a metal highly reflective to the wavelength of laser energy to be used, such as highly pure gold or silver, or metal end piece 23 can be made of a material such as medical grade stainless steel, which is plated with a highly reflective metal, such as highly pure gold or silver with a thickness of about 5 thousandths of an inch or more, or coated with a dielectric highly reflective to the wavelength of laser energy to be used, as known in the art.

Alternatively, an insert (not separately shown) with a thickness of about 10 to 20 thousandths of an inch or more of a metal highly reflective to the wavelength of laser energy being used, such as highly pure gold or silver, may be force-fitted or attached by an adhesive, or both, in a recess (not separately shown) in the distal end of the cavity 22 in metal end piece 23.

Polished copper, brass, aluminum or stainless steel, which cost less than gold or silver, may also be used. However, stainless steel is not a highly efficient reflector, and copper and aluminum are not as reflective as gold or silver and are subject to tarnish and/or oxidation, which reduced their reflectivity.

95.5% pure Silver is about 97% reflective at wavelengths of about 500 to 2400 nm, and about 95.5% reflective at 430 nm. 95.5% pure Gold is less than 50% reflective below wavelengths of 500 nm, 81.7% reflective at 550 nm, 91.9% reflective at 600 nm, 95.5% reflective at 650 nm and about 97% reflective at 700 nm and longer wavelengths. Highly pure platinum is extremely expensive and is only 71.4% to 81.8% reflective at wavelengths of 500 to 2000 nm and is 88.8% reflective at 3000 nm. Highly pure silver is preferred, because it is highly reflective and is considerably less expensive than gold or platinum.

However, for greater durability, a lower cost of manufacture and resistance to erosion by the emission of laser energy, metal end piece 23 is preferably made entirely of at least 90% pure gold or silver, preferably of very pure silver with a purity of about 95.5%. For comparison, “Sterling” silver is 92.5% pure.

The second embodiment of side firing device 10 of the present invention is shown in FIG. 2. In this embodiment, distal end 16 of hollow cannula 15 is shaped like the distal end of a double beveled syringe needle, which cuts rather than making a puncture or hole through the skin, hastening healing and reducing bleeding and the risk of an infection. To prevent tissue from lodging in the opening at distal end 16 of cannula 15, plug 27 of an adhesive or other material, preferably heat resistant to any stray laser energy, may be used to fill distal end 16 of cannula 15, as known in the art.

Distal end 16 of hollow cannula 15 can also be blunt, round, conical or any other desired shape, as the use of a sharp or needle-like device within a patient requires imaging during its use and great care by the surgeon.

Buffer coating 21 and any optional polymer cladding have been removed from the distal end portion of optical fiber 12, and the distal end of optical fiber 12 has been ground and polished into beveled, distal end surface 28 at an angle of about 35° to 45°. The beveled, distal end portion of optical fiber 12 is sealingly encased within hollow, closed-ended capillary tube 29, which creates air pocket 30 opposite beveled, distal end surface 28 of optical fiber 12. Air pocket 30 has a lower refractive index than that of the core of optical fiber 12, which is necessary for total internal reflection or “TIR” of laser energy at double the bevel angle of distal, beveled end surface 28, according to Snell's Law.

According to common wisdom in the medical laser field, the most effective bevel angle of an optical fiber for total internal reflection of laser energy is 37°. Contrary to common wisdom, however, distal end surface 28 of optical fiber 12 is preferably beveled at an angle of about 40° to 41°, which we have discovered by testing various bevel angles at 1° intervals, to be the most efficient bevel angle of an optical fiber for total internal reflection of laser energy at relatively high power levels.

If beveled, distal end surface 28 of optical fiber 12 is ground and polished at an angle less than 40°, the laser energy will be less optimally reflected and more scattering of laser energy will occur. If distal end surface 28 of optical fiber 12 is beveled at an angle greater than 42°, the transmission of laser energy will be substantially lower.

Capillary tube 29 typically has a wall thickness of 500 microns or more, as it may be eroded during use, causing device 10 to fail. The proximal end portion of closed-ended capillary tube 29 may be fixedly and sealingly attached to the bared distal end portion of optical fiber 12 by thermal fusion (not separately shown) or by adhesive 26, neither of which extend into the area of laser energy emission from beveled, distal end surface 28 of optical fiber 12. While not preferred, if capillary tube 29 is fused to optical fiber 12 near beveled, distal end surface 28 of optical fiber 12, care must be taken to avoid deforming beveled distal end surface 28 of optical fiber 12 by exposure to high glass fusing temperatures.

FIG. 3 illustrates the third embodiment of side firing device 10 of the present invention. In this embodiment, no capillary tube 29 is utilized to sealingly encase the beveled, distal end surface 28 of optical fiber 12. As a result, no air pocket is created opposite beveled, distal end surface 28 of optical fiber 12.

Laser energy at wavelengths of 1400 to 1500 nm and 1800 to 11,000 nm are highly absorbed by aqueous liquids, such as sterile saline or water, which are commonly used as an irrigation fluid in endoscopic procedures. If ten or more watts of laser power at these wavelengths is transmitted through optical fiber 12, such wavelengths of laser energy cause a steam and/or gas bubble (not separately shown) to form, with each pulse of laser energy, opposite beveled, distal end surface 28 of optical fiber 12, from the vaporization of the aqueous irrigation liquid, blood, other body fluids and/or tissue.

The refractive index of the steam and/or gas bubble opposite beveled, distal end surface 28 of optical fiber 12 is sufficiently lower than the refractive index of the quartz or fused silica core of optical fiber 12, to enable the laser energy to be totally internally reflected from beveled, distal end surface 28 of optical fiber 12, laterally from the axis of optical fiber 12 at an angle of 80° to 82°, as shown by arrows 19, according to Snell's Law, and the balance of the pulse of laser energy passes through the stream and/or gas bubble to the Target Tissue. Consequently, no capillary tube 29 must be disposed over the 41° to 42° beveled, distal end surface 28 of optical fiber 12 to create an air interface and TIR.

However, laser energy at 300 to 1400 and 1500 to 1800 nm cannot be used through device 10 of this third embodiment of the present invention, as such wavelengths are not highly absorbed by water and no steam and/or gas bubble with a refractive index tower than the core of optical fiber will be formed, and the laser energy will be emitted straight-ahead.

As shown, distal end 16 of cannula 15 is pointed or conically shaped. As mentioned above, the use of a pointed or sharp-ended cannula in a Patient entails significant risk and should be used under endoscopic, ultrasound or x-ray viewing.

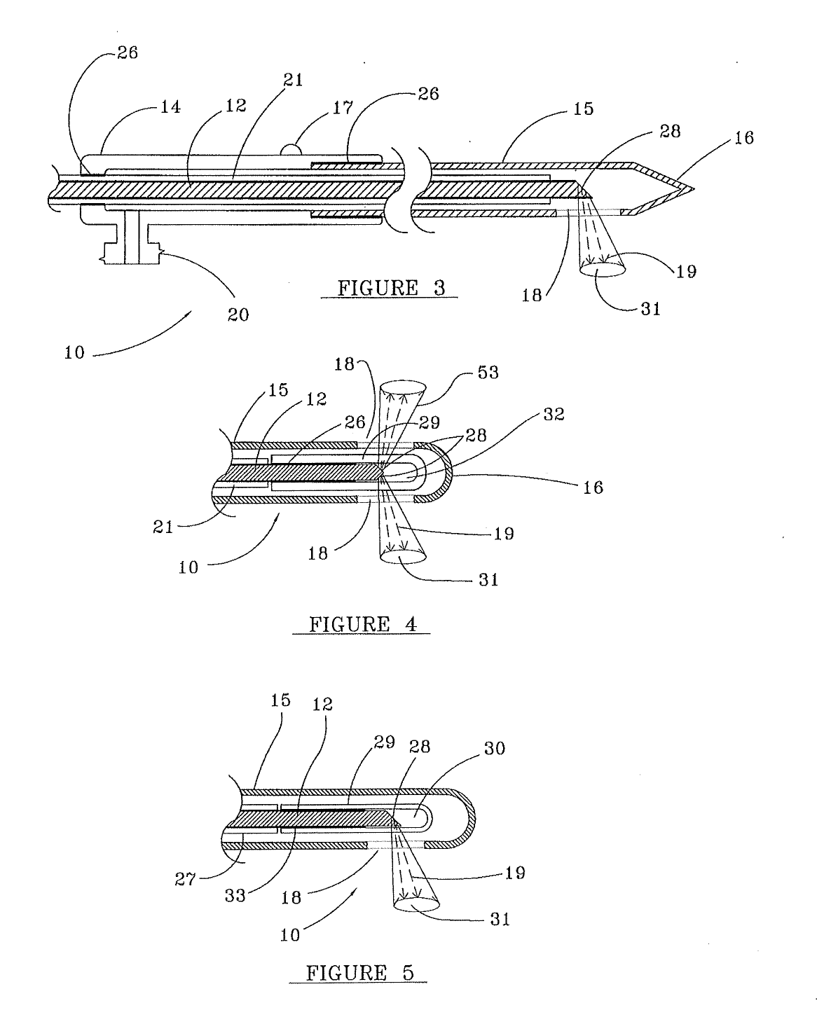

FIG. 4 illustrates side firing device 10 in which the distal end of optical fiber 12 is beveled into a chisel like shape, with each distal, beveled end surface 28 at an angle of 40° to 42° from the axis of optical fiber 12. The proximal end of capillary tube 29 is fixedly attached to the bared distal end surface of optical fiber 12, buffer coating 21 and any polymer cladding (not separately shown) having earlier been removed from the distal end portion of optical fiber 12.

Capillary tube 29 creates air pocket 30 opposite both distal beveled end surfaces 28 of optical fiber 12, necessary for total internal reflection (TIR) of laser energy, according to Snell's Law. As indicated by arrows 19, laser energy is simultaneously emitted from both ports 18 in cannula 15 exits at an angle of about 80° to 82° from the axis of optical fiber 12, simultaneously creating laser energy spot areas 31.

In this embodiment, to achieve the same effect on a Target Tissue, the level of laser energy must be doubled.

FIG. 5 illustrates a further improved embodiment of side firing device 10 of FIG. 2. In this embodiment, bared optical fiber 12 is fixedly and sealingly encased within a distally closed-ended capillary tube 29, which has a substantially thinner wall thickness than the typical 500 micron or larger wall thickness of capillary tube 29 shown in FIG. 2. The wall thickness of capillary tube 29 in this embodiment is preferably about 350 microns or less.

This reduces the amount of cylindrical lensing that occurs and converges the divergent output of laser energy from beveled, distal end surface 28 of optical fiber 12 at a closer point, providing an effectively wider angle of divergence at a given distance from laser energy emission port 18, as illustrated by arrows 19. This results in a significantly larger laser energy spot area 31 on or within a Target Tissue (not separately shown) than laser energy spot area 31 shown from side firing device 10 of FIG. 2.

However, this embodiment of the present invention is preferably used at low levels of laser energy. Side Firing device 10 of FIG. 5 should not be used to Treat a Medical Condition of a Patient which requires the emission of a very high level of laser energy for a substantial period of time, such as 40 to 100 watts for 10 minutes or longer, as thinner capillary tube 29 is more likely to be degraded by hydrothermal erosion and laser energy back reflected from the Target Tissue, causing device 10 to fail.

Hydrothermal erosion is created by the formation of a steam bubble, when each pulse of laser energy at wavelengths of 1400 to 1500 and 1800 to 11,600 nm is emitted, and a powerful acoustic shock wave is created by the collapse of the bubble, which can erode capillary tube 29.

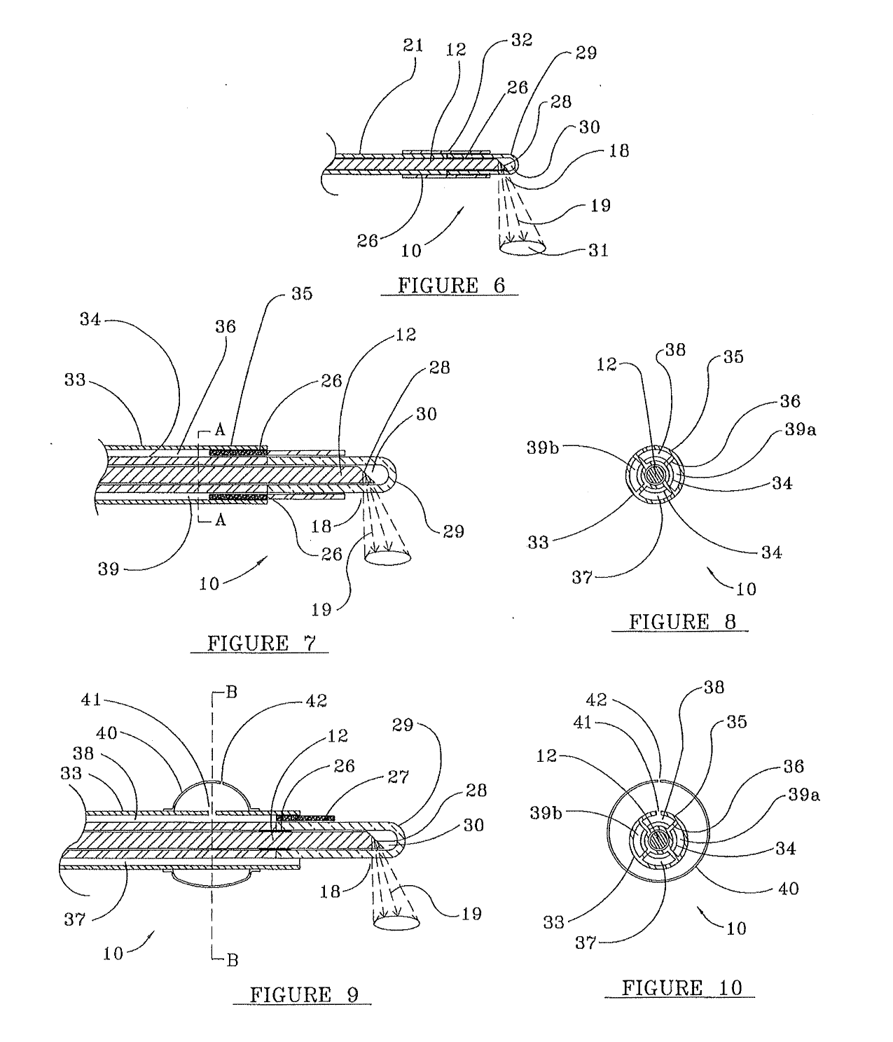

FIG. 6 illustrates a sixth embodiment of side firing device 10 of the present invention. Side firing device 10 is made with an unusually small optical fiber, with a core diameter of 350 microns or smaller, and can be bent at an angle of up to 90° or more when used, for example, through a conventional guiding catheter (not separately shown) to access from the internal aorta, at an angle of up to 180°, the left ventricle of the heart (not separately shown), to shrink the chordae tendinae of a prolapsed mitral, aortic or tricuspid valve to treat the valve's prolapse, sometimes called regurgitation or leakage. However, optical fibers with a core diameter of 500 microns or larger may not be sufficiently flexible to be used through such a guiding catheter.

Common wisdom in the laser field that is only optical fibers with core diameters of 500 microns or larger can be effectively used to transmit up to 100 watts or more of laser power, and have a sufficient surface to be beveled to effectively reflect laser energy at an angle of 70° to 90°. Contrary to common wisdom, however, by testing optical fibers of successively smaller diameter, we discovered that optical fibers with a core diameter of 350 microns or smaller could be effectively beveled and used with appropriate cladding materials through bends of up to 90° or more with up to about a 95% laser energy transmission efficiency, provided the bend radius is not less than 1 to 1.5 cm, as will be explained later.

As a result, as seen in FIG. 6, we created what we believe is the smallest diameter side firing device 10 ever made, with an O.D. of 1.4 mm or less, compared to prior art side firing devices 10 with an O.D. of 2 mm to 2.5 mm, enabling this smaller diameter side firing device 10 to be used in arteries, veins, bronchi, ducts, hollow organs, body orifices and surgically created passageways with an I.D. of 1.6 mm or smaller, which may optionally be cannulated.

As seen, optical fiber 12 has a core diameter of 365 microns, whose distal end surface 28 has been beveled at an angle of 40 to 41° from the axis of optical fiber 12. Buffer coating 21 and any optional polymer cladding (not separately shown) has been removed from the distal end portion of optical fiber 12, and capillary tube 29 fixedly and sealingly encases the distal end portion of optical fiber 12, as described above, creating air pocket 30 to enable total internal reflection of light to occur through port 18, as shown by arrows 19.

For use at relatively high laser energy levels, as shown, capillary tube 29 can have a wall thickness of about 500 microns. For use at relatively lower levels of laser energy, capillary tube 29 can have a wall thickness of 350 microns or less, as shown in FIG. 4.

The proximal end portion of capillary tube 29 can be fixedly attached to bared optical fiber by thermal fusion (not separately shown), by adhesive 26 or both. Adhesive 26 is preferably made of a material with a high melting point, which meets USP Class VI specifications for use in medical devices and which is substantially transparent to the wavelengths of laser energy commonly used in medical procedures, such as KTP, diode, Nd:YAG, Thulium:YAG and CTH:YAG or Holmium lasers, so as not to absorb laser energy and melt, allowing capillary tube 29 to move with respect to optical fiber 12 and be dislodged therefrom.

Adhesive 26 has a high melting point, and is substantially transparent to and does not absorb the wavelengths of laser energy commonly used in medical procedures, such as 532 mm KTP, 980 mm diode, 1046 nm Nd:YAG or 2100 nm CTH:YAG laser energy, not absorbing more than an average of 6% of such laser energy. Preferably, the adhesive is an optically transparent, two-component epoxy adhesive.

As a safety measure, heat shrinkable tubing 32 is shrunk over the distal end portion of buffer coating 21 and the proximal end portion of capillary tube 29, terminating before laser energy emission port 18. Adhesive 26 can also be optionally used to fixedly attach heat shrinkable tubing 32 in place, as an additional safety measure, to help prevent the accidental separation of capillary tube 29 from optical fiber 12.

FIG. 7 illustrates the seventh embodiment of device 10 of the present invention. In this embodiment, flexible plastic, round, hollow, doubled-walled, multi-channel tube 33 extends from about the distal end of or within the distal end of handpiece 14 (not separately shown) over optical fiber 12 and, as shown, terminates just before the proximal end of heat shrunk tubing 32.

Round, hollow, double-walled, multi-channel tube 33 consists of round inner wall 34 and round outer wall 35. The I.D. of inner wall 34 of tube 33 is just slightly larger than the O.D. of optical fiber 12. To space inner wall 34 apart from outer wall 35, tube 33 is extruded with two or more longitudinally extending ribs 36 (not separately shown in FIG. 8). Preferably four ribs 36 are extruded, creating four channels 37, 38, 39(a) and 39(b) (not separately shown), as described below in FIGS. 8-12.

FIG. 8 illustrates the construction of flexible, round, hollow, double-walled, multi-channel tube 33 of device 10 at a plane A-A of FIG. 7. Inner wall 34 of tube 33 is circular with an I.D. just slightly larger than that of optical fiber 12 of the devices of FIGS. 1-4, the relatively smaller diameter optical fiber 12 of FIG. 6 described above, and the diameter of optical fiber 12 of FIGS. 9-12 described below.

In this embodiment, for example, four ribs 36 extend longitudinally through and separate inner wall 34 from outer wall 35 of tube 33, with ribs 36 preferably located at 2, 4, 8 and 10 o'clock, creating channels 37, 38, 39(a) and 39(b).

Channel 37 may be in fluid communication with fluid passageway in handpiece 14 and luer fitting 20 (neither of which are separately shown), and a sterile biocompatible fluid, such as saline or water, can be infused through channel 37 to clean and cool the laser energy emitting surface of capillary tube 29 and cool the Target Tissue.

FIG. 9 illustrates the eighth embodiment of device 10 of the present invention. In this embodiment, double walled, hollow tube 33 extends from about the distal end or within the distal end of handpiece 14 (not separately shown), over optical fiber 12 and heat shrunk tubing 32 and co-terminates with the distal end of heat shrunk tubing 32, proximal to the area of laser energy emission from capillary tube 29, as shown by arrows 19.

Balloon 40 eccentrically encases a portion of the distal end portion of hollow, double-walled tube 33. The wider portion of eccentric balloon 40 presses the laser energy emitting surface of capillary tube 29 closer to the Target Tissue and minimizes the loss of laser energy in vaporizing any intervening aqueous irrigation fluid. Irrigation fluid infused through channel 37 also forces bodily liquids (not separately shown) away from the laser energy emission area of capillary tube 29, as shown by arrows 19.

A biocompatible irrigation fluid, such as sterile saline or water, may also be infused through channel 38 and exits vent 41 in outer wall 35 (FIG. 10) to inflate balloon 40. In this embodiment, balloon 40 has one or more vent holes 42. When the irrigation fluid is infused through channel 38 to inflate balloon 40, one or more vent holes 42 enable air to be purged from channel 38 and escape from balloon 40. When irrigation fluid is seen exiting tiny hole or holes 42, the operator knows the air has been purged from channel 38.

In this embodiment, channels 39(a) and 39(b) are not used, and the proximal ends of channels 39(a) and 39(b) and the distal ends of channels 38, 39(a) and 39(b) are closed by plugs 27 of adhesive 26 or other material known in the art (not separately shown).

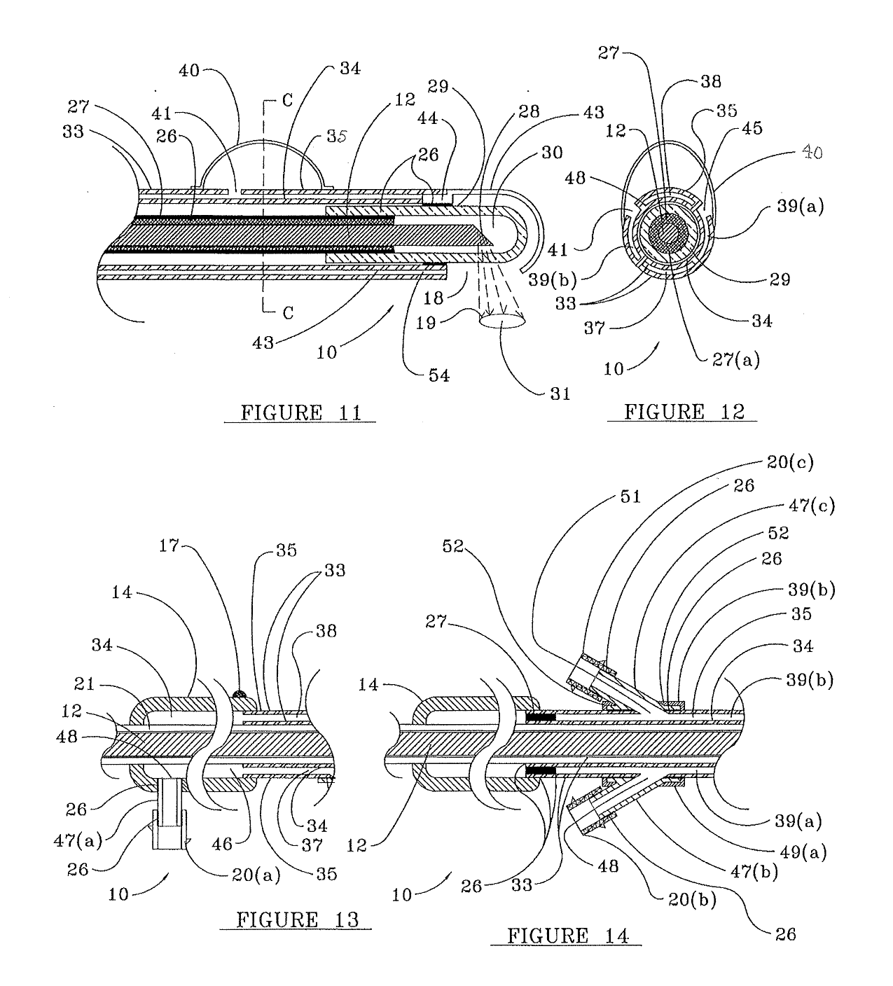

Balloon 40 can also be back-mounted to force the energy emission port 18 of device 10 close or closer to the Target Tissue, as described in FIG. 11, below.

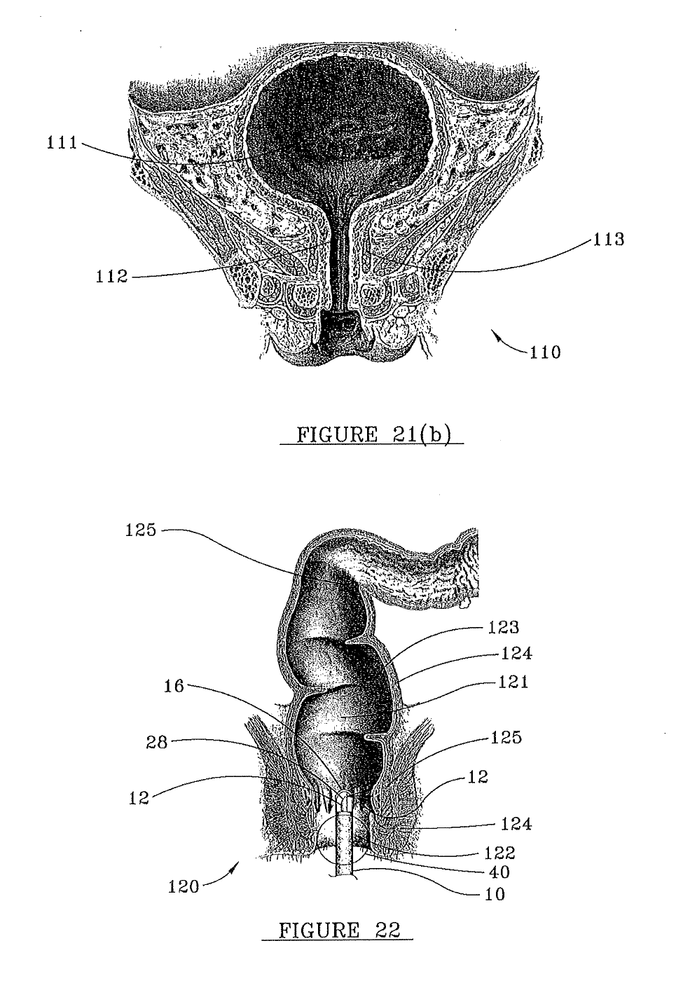

As described in FIG. 22 below, balloon 40 can also be concentric to center device 10 in a body orifice, hollow organ or surgically created passageway and insure an equal amount of laser energy will be emitted to the inner surface of the orifice, hollow organ or passageway at each area of laser energy emission, where this effect is desired.

FIG. 10 illustrates the construction of double-walled, hollow tube 33 of device 10 at plane B-B of FIG. 9. In this embodiment, outer wall 35 of double-walled, hollow tube 33 has vent 41, allowing a sterile, biocompatible fluid, such as saline or water, to be infused through channel 38 and exit through vent 41 to inflate eccentric (or concentric or back-mounted) balloon 40 which encases the portion of device 10 proximal to its laser energy emitting surface.

FIG. 11 illustrates the ninth embodiment of device 10 of the present invention. In this embodiment, inner wall 34 of double-walled, hollow tube 33 is circular to accept capillary tube 29 sealingly encasing optical fiber 12, which are disposed eccentrically within double-walled, hollow tube 33, by a relatively thicker walled plug 27, versus that of a relatively thinner walled plug 27(a) (FIG. 12), positioning the laser energy emitting surface of capillary tube 29 closer to the Target Tissue. Vent 41 in outer wall 36 allows a sterile, biocompatible fluid, for example, saline or water, to be infused through channel 38 and vent 41 to inflate balloon 40.

Balloon 40 is mounted on the back side of hollow, double-walled tube 33, opposite the side of device 10 from which laser energy is emitted from capillary tube 29, as shown by arrows 19. The inflation of balloon 40 forces side firing device 10 close to the Target Tissue, and the infusion of fluid through channel 37 forces blood away from the path of laser energy emission.

FIG. 12 further illustrates the construction of device 10 at plane C-C of FIG. 11. In this embodiment, fluid infused through channel 38 and vent 41 in outer wall 35 to inflate balloon 40, exits balloon 40 through vent 45 in outer wall 35 into return channel 39(a), through a luer fitting and flows to a drain or a collection bottle (not separately shown).

Alternatively fluid return channel 39(a) can empty into a plastic tube which can be clamped shut, as known in the art, when balloon 40 has been inflated, and which can be unclamped and a vacuum applied to empty balloon 40 and channels 38 and 39(a) when the procedure has been completed to enable device 10 to be safely removed from the patient. The distal ends of channels 38, 39(a) and 39(b) remain closed by cylindrical plugs 27.

Optional end cap 43, which may be made of metal or a rigid plastic, as shown, is rounded to provide an atraumatic distal end of device 10. End cap 43 may be blunt, sharp, pointed or of any other desired shape. Circular flange 44 of end cap 43 is fixedly attached between outer wall 35 and inner wall 34 of hollow, double-walled tube 33 by adhesive 26 and effectively plugs the distal ends of channels 38, 39(a) and 39(b).

FIG. 13 illustrates how luer fitting 20(a) enables a sterile, biocompatible fluid to pass through luer fitting 20(a) and opening 48 into passageway 46 in handpiece 14 and flow through channels 37 and 38 of double-walled, hollow tube 33 to cool and clean debris from capillary tube 29 (not separately shown) and cool the Target Tissue.

Luer fitting 20(a) is fixedly attached to luer tube 47(a) by adhesive 26. Luer tube 47(a) is attached to handpiece 14 by adhesive 26, and is in fluid communication through opening 48 with passageway 46 in the body of handpiece 14.

As seen in FIG. 14, luer fittings 20(b) and 20(c) of device 10 are in fluid communication with double-walled, hollow tube 33, which has four channels, 37 and 38, as shown in FIGS. 13, and 39(a) and 39(b), as shown in FIG. 14. These channels are created by four ribs 36 extending longitudinally through and separating inner wall 34 from outer wall 35 of double-walled, hollow tube 33.

Fluid also passes through luer fitting 20(b), which is fixedly attached by adhesive 26 to luer tube 47(b), and flows through opening 48 into channel 39(a) of hollow, double-walled tube 33 and vent 50 (as seen in FIG. 12) in outer wall 35 of double-walled tube 33 to inflate balloon 40 (not separately shown). Excess fluid used to inflate balloon 40 exits balloon 40 through vent 45 (as seen in FIG. 12) in outer wall 35 of double-walled tube 33 and flows out through channel 39(b) and luer fitting 20(c) to a drain (not separately shown).

Luer tubes 47(b) and 47(c), whose distal ends are cut at an angle or bias, as shown, are attached by adhesive 26 to outer wall 35 of double-walled tube 33.

Luer tubes 47(b) and 47(c) are extruded with circular flanges 49(a) and 49(b), respectively, which are fixedly attached by adhesive 26 or other adhesive known in the art to outer wall 35 of hollow, double-walled tube 33 over openings 48 and 51, respectively, in outer wall 35, and are in fluid communication with channels 39(a) and 39(b), respectively. Plugs 27 close the proximal ends of channels 39(a) and 39(b), which may comprise adhesive 26 or the like.

Since luer fittings 20(b) and (c) are attached to outer wall 35 of double walled, hollow tube 33, instead of being attached to handpiece 14, luer fittings 20(b) and 20(c) and luer tubes 47(b) and 47(c), respectively, do not interfere with the surgeon's handling of handpiece 14 of side firing device 10.

To provide extra support to luer tubes 47(a) and 47(b) and to flanges 49(a) and 49(b), optionally, metal or rigid plastic collar 52 may be attached to outer wall 35 of double-walled tube 33, flanges 49(a) and 49(b) and the bottom, proximal portion of luer tubes 47(b) and 47(c) by adhesive 26.

For ease of use, luer tubes 47(a) and 47(b) and luer fittings 20(b) and 20(c) are disposed on outer wall 35 of double-walled, multichannel tube 33 a desired distance proximally from the proximal end of handpiece 14. The distal ends of channels 39(a) and 39(b), proximal to luer tubes 47(b) and 47(c), are closed by plugs 27 of adhesive 26 or other adhesive known in the art.

Any other number of ribs 36 may be used, creating any desired number of fluid channels, and ribs 36 may be positioned at any points, as desired, so long as none are in the path of laser energy emission from capillary tube 29 (not separately shown).

As mentioned earlier, 350 micron or smaller, thinner walled capillary tube 29 shown in FIG. 6 can be utilized in any of the embodiments of the present invention shown in FIG. 2 or 3. If side firing device 10 is to be used to emit a low level of laser power in a non-aqueous environment or in the absence of cooling liquid spray, it should be used, for example, at about 0.01 to 3 watts. Likewise, capillary tubes 29 with a wall thickness greater than 350 microns, for example, about 400 to 600 microns, can be used in side firing devices 10 if laser energy at higher levels is to be used, for example, at about 20 to 100 watts.

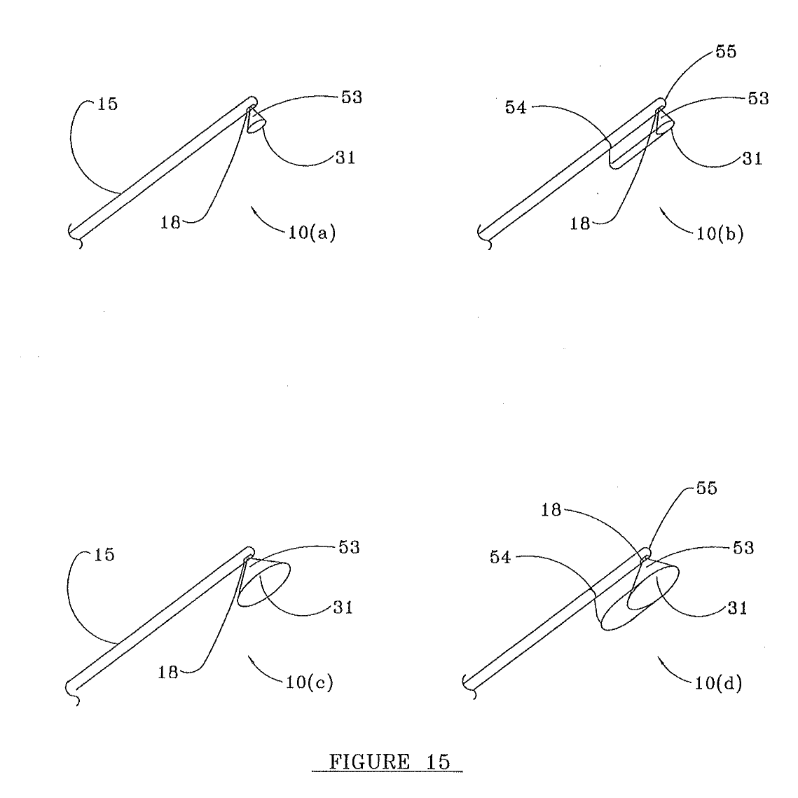

FIG. 15 illustrates four laser devices 10(a-d). The energy emission pattern 53 and laser energy spot area 31, resulting from positioning laser energy emission port 18 of side firing device 10(a), without moving device 10(a) or port 18, while laser energy is emitted at a desired energy level for a desired period of time, in a desired direction.

FIG. 15 also illustrates larger laser energy emission pattern 53 and larger laser energy spot area 31 resulting from positioning device 10(c) and Moving, by repetitively advancing and withdrawing side firing device 10 and laser energy emission port 18 at a desired rate of movement, from first point 54 to second point 55, while laser energy at a desired level for a desired period of time is emitted in a desired direction. The rate of Movement, the level of laser energy emitted and the time period of such emission is dependent, in the physician's discretion, upon the volume and depth of the Target Tissue to be Treated or the Interruption or Altering effect desired to be achieved on the Target Tissue.

FIG. 15 also illustrates the laser energy emission pattern 53 and laser energy spot area 31, resulting from positioning side firing device 10(c) and repetitively Rotating device 10 and laser energy emission port 18 through an arc of about 90 to 120°, while laser energy is emitted at a desired level and for a desired period of time, in a desired direction, at a rotation rate of about 0.5 to 2 seconds per cycle, preferably about 1 cycle each second, enabling the surgeon to mentally count, one thousand, two thousand, etc. per arc during the laser energy emission period.

FIG. 15 also illustrates the larger laser energy emission pattern 53 and larger laser energy spot area 31 obtained by combining the above described Moving and Rotating processes of device 10(d), together or in any desired order or sequence, and Sweeping the laser beam, at a desired level of laser energy, for a desired period of time, while laser energy is emitted in a desired direction, at a desired rate of Movement and Rotation from first point 54 to second point 55, to Alter a large area or swath of Target Tissue.

As seen in devices 10(a-d) of FIG. 15, laser energy diverges as it exits port 18, and the laser beam is narrow close to the laser energy's exit point from port 18. The benefit of combining the Moving process of device 10(c) with the Rotation process of device 10(d) in the Sweeping process described above as a wide area or swath of Target Tissue is irradiated, resulting in a more uniform shrinkage of a Target Tissue to Treat a Medical Condition of a Patient.

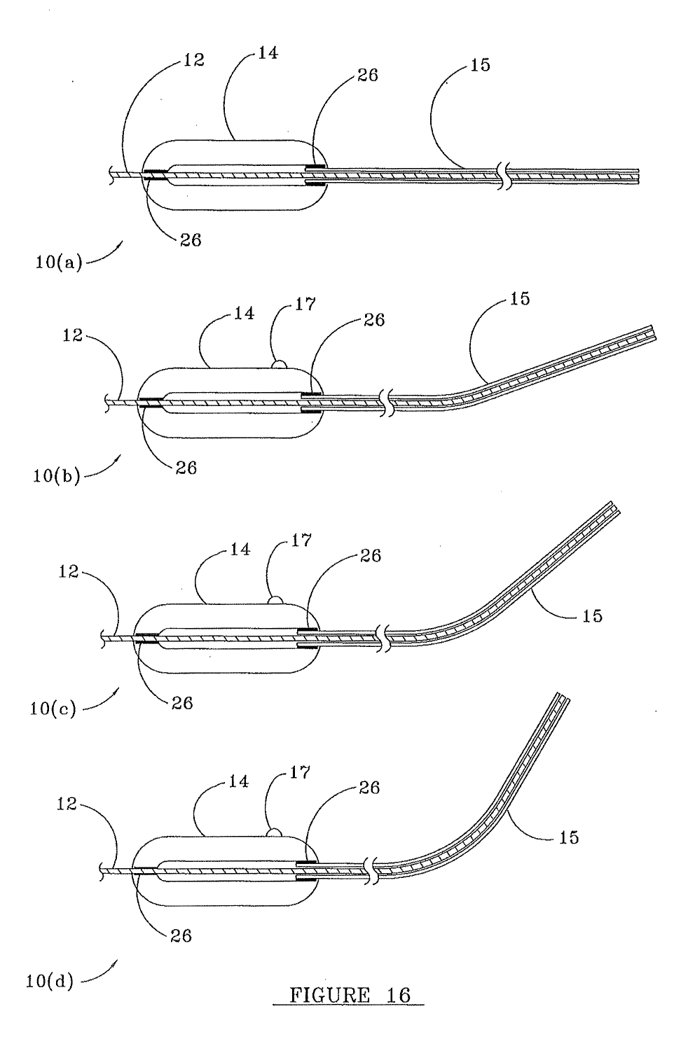

FIG. 16 illustrates four prior art laser energy delivery devices 10(a)-(d). The four devices 10(a)-(d) each contain optical fiber 12, which passes through handpiece 14, is fixedly attached within the proximal or distal end of handpiece 14, closely fits within (or is fixedly attached by adhesive 26 to) the interior of rigid plastic or metal cannula 15, which is preferably made of medical grade stainless steel.

Optical fiber 12 co-terminates at about the distal end of cannula 15, whose proximal end is fixedly attached within the distal end of handpiece 14. In each of devices 10(a)-(d), laser energy is emitted from the flat, distal end of optical fiber 12 straight ahead at an angle of 0° from the axis of the optical fiber.

Alternatively, optical fiber 12 can be removably attached to the proximal end of handpiece 14 by a compression nut (not separately shown) as known in the art, enabling optical fiber 12 to be extended distally from the distal end of cannula 15 for cleaning and, if needed, clipping and cleaving to remove any deformed portion of optical fiber 12.

As can be seen, cannula 15 of device 10(a) is straight, to emit laser energy straight ahead at an angle of 0° from the axis of cannula 15. Cannula 15 of device 10(b) has a bend proximal to its distal end, as shown, at an angle of 20° from the axis of the main body of cannula 15. Cannula 15 of device 10(c) has a bend proximal to its distal end, as shown, at an angle of 40° from the axis of the main body of cannula 15, and cannula 15 of device 10(d) has a bend proximal to its distal end at an angle of 60° from the axis of the main body of cannula 15. Cannula 15 may also have a bend proximal to its distal end of 10°, 30°, 50° or any other desired angle from the axis of the main body of cannula 15.

However, depending on the core diameter of optical fiber 12, the level of laser energy to be transmitted through optical fiber 12 and the temperature at which the cavity or lasing element of the laser is maintained, the radius of the bend must not be less than a certain radius, or leakage of laser energy through the quartz or fused silica cladding (not separately shown), which surrounds optical fiber 12, may occur. The cladding may contain a dopant, such as fluorine to lower its refractive index.

Escaping laser energy may cause cannula 15 to overheat and cause damage to cannula 15 and the instrument channel and optics of an endoscope (not separately shown), through which cannula 15 may be used. For example, if the cavity or lasing element (not separately shown) of the source of laser energy 11 is cooled by a heat exchange device (not separately shown) to a temperature of about 2 to 5° C., if optical fiber 12 has a core diameter of 365 microns and 10 watts of Holmium laser energy is to be transmitted through optical fiber 12, the bend radius must not be less than 1 cm.

If the cavity or lasing element (not separately shown) of the source of laser energy 11 is cooled by a chiller (not separately shown) to a temperature close to freezing, about 0° C., if optical fiber 12 has a core diameter of 365 microns and 10 watts of Holmium laser energy is to be transmitted through optical fiber 12, the bend radius must not be less than 1.5 cm. As a result, bends in the distal end portion of cannula 15 must be made at a shallow angle.

While there is no button 17 on handpiece 14 of the 0° emitting or straight cannula 15, cannulas 15 bent at angles of 20°, 40°, 60°, as shown, or at any other desired angles, have button 17 on the side of handpiece 14 opposite from the direction of the bend, so the surgeon knows in what direction cannula 15 is being extended and the direction of laser energy emission. Button 17 should be raised and have a color different from that of handpiece 14, so it can be seen and be recognized by tactile feel by the surgeon.

Devices 10(a)-(d) of FIG. 16 may be used where it is impractical to deliver laser energy from any of the side firing devices 10 described in FIG. 1-5, 7, 9 or 11. While Devices 10(a-d) of FIG. 16 are prior art devices, their use in the Stationing, Moving, Rotating and Sweeping methods, described above, to shrink a Target Tissue to treat a Medical Condition are novel and unique to the practice of the present invention.

A disadvantage of prior art devices 10 of FIG. 16 is they have no provision for delivering a sterile, biocompatible fluid to cool and clean the distal end of optical fiber 12 and cool the Target Tissue, as the devices 10 of FIG. 16 are typically used in an aqueous Environment, such as sterile water or saline. As a result, if side firing devices 10 of FIG. 1-5, 7, 9 or 11 or devices 10 of FIG. 16 are used in air or a CO₂ Environment, for example, in a laparoscopic or endoscopic procedure, a much lower level of laser power, 0.05 to 10 watts, preferably 0.1 to 3 watts, should be used to prevent excessive heating, coagulation or charring of the Target Tissue and thermal damage to adjacent tissues.

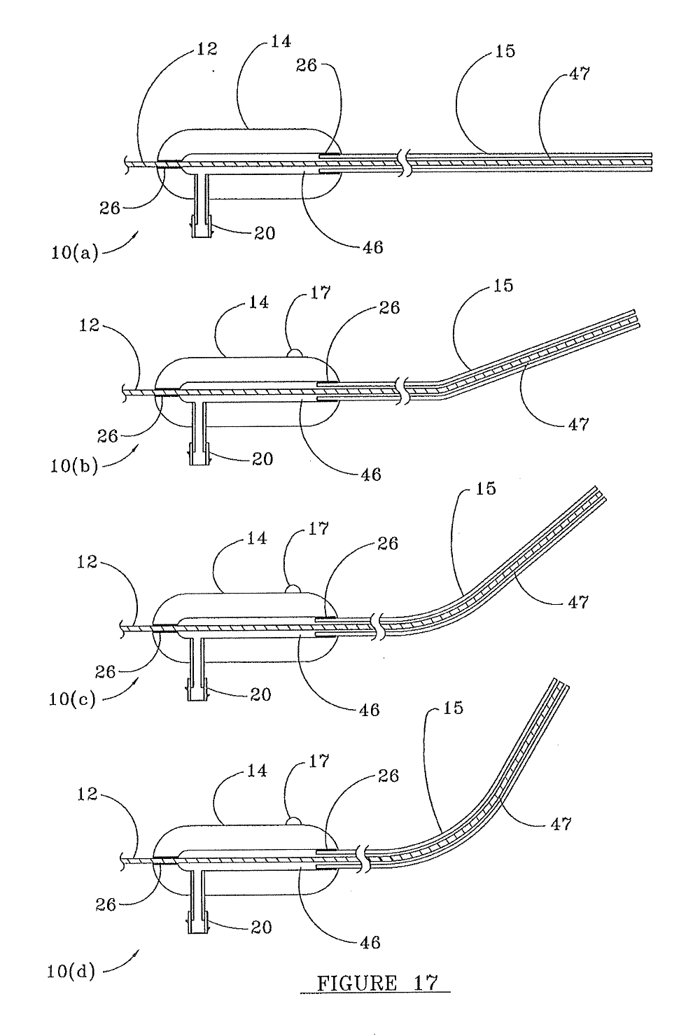

FIG. 17 illustrates the solution to the problem described above with respect to FIG. 16, and represents an improved version of prior art devices 10(a)-(d) of FIG. 16. In this embodiment of the present invention, cannula 15 of devices 10(a-d) can be made of a thin, rigid metal, preferably medical grade stainless steel, for use under x-ray guidance through a body orifice, hollow organ, surgically created passageway or in a laparoscopic procedure, positioned and guided by an endoscope (not separately shown), through which cannula 15 may be inserted, or the endoscope may be inserted through a separate puncture.

Alternatively, cannula 15 of devices 10(a-d) can be made of a thin, flexible biocompatible plastic (not separately shown), for use through a flexible, articulated endoscope (not separately shown) or an endoscope of which the distal 5 to 15 cm may be bent or articulated at a described angle by wires (not separately shown) extending from a handpiece (not separately shown) to the distal end of the endoscope.

Preferably, devices 10(a)-(d) are made of a flexible memory metal, such as nitinol, an alloy of about 56% nickel and about 44% titanium by weight, such as those made by Memry, Inc. of Bethel, Conn., which are heat treated to “remember” their heat treated shape, to which they return after being straightened-out, for example, by passing through the instrument channel of an endoscope. Some semi-rigid plastics may also retain the memory of their initially molded shape, and can be used in devices 10(a)-(d).

In the embodiments of devices 10(a)-(d) of the present invention shown in FIG. 17, luer fitting 20 is fixedly attached within the wall of handpiece 14 and is in fluid communication with hollow passageway 46 in handpiece 14, as described in FIG. 14, and is in fluid communication with the space between the exterior of optical fiber 12 and the interior of cannula 15, creating fluid channel 47, enabling a sterile, bio-compatible fluid, such as saline or water, to be infused through fluid channel 47 to clean and cool the distal end of optical fiber 12 and to cool the Target Tissue, concomitantly with the delivery of laser energy.

Optical fiber 12 is fixedly attached within the proximal end of handpiece 14, the proximal end of cannula 15, is fixedly attached within the distal end of handpiece 14 and luer fitting 20 can be fixedly attached to and in fluid communication with passageway 46 in handpiece 14, and fluid channel 47, as shown in FIG. 17. Likewise, collar 52 as described in FIG. 14, can be used to support and prevent damage to luer fitting 20, if luer fitting 20 is attached to cannula 15, as described above.

As can be seen, devices 10(a)-(d) of FIG. 17 have bends at the same angles as devices 10(a)-(d) of FIG. 16. Again, such bends and others at any other desired angles may be employed, subject to the bend radius limitation described above.

The embodiments of devices 10(a)-(d) of the present invention shown in FIG. 17 can be used in the positioning, Moving, Rotating and/or Sweeping processes described above, individually or in any combination or sequence. The use of devices 10(a)-(d) shown in FIG. 17 are beneficial in instances where the use of side firing devices 10 of the present invention shown in FIG. 1-5, 7, 9 or 11 is difficult or impractical.

Alternatively, luer fitting 20 may be attached to cannula 15, distal to handpiece 14, as described in FIGS. 13 and 14, and luer fitting 20 may optionally be supported by collar 52, as described in FIG. 14.

The “working” length of devices 10 of FIG. 1-5, 7, 9, 11, or 17 are typically 15 cm to 80 cm in length, extending distally from the distal end of handpiece 14.

All of the side firing devices 10 of the present invention described in FIG. 1-5, 7, 9 or 11 may be utilized with or without rigid plastic or metal cannula 15, with or without double-walled, hollow tube 33, or with or without collar 52 to support luer fitting 20. These appurtenances, and the thinner walled capillary tube 29 of FIG. 4, are to enable any or all of the above embodiments of the present invention to better accomplish their desired purpose.

While the aforementioned laser energy emission patterns 53 and laser energy spot areas 31 are described as resulting from the emission of laser energy, any other Thermal Energy delivery device may be used in any of the above-described processes of positioning, Moving, Rotating and/or Sweeping the Thermal Energy delivery device, alone or in any desired combination and in any desired sequence or order, to shrink a Target Tissue to Treat a Medical Condition of a Patient.

The uses of device 10 of FIGS. 1-5, 7, 9, 11, 16 and 17 of the present invention are shown in some of FIGS. 21-25 below and are intended to illustrate the methods of use of this invention in Treating a Medical Condition of a Patient. All of devices 10 illustrated in FIGS. 1-5, 7, 9, 11, 16 and 17 have a common purpose, namely to efficiently and uniformly shrink Target Tissues to Treat a Medical Condition, when used by the methods of use described above.

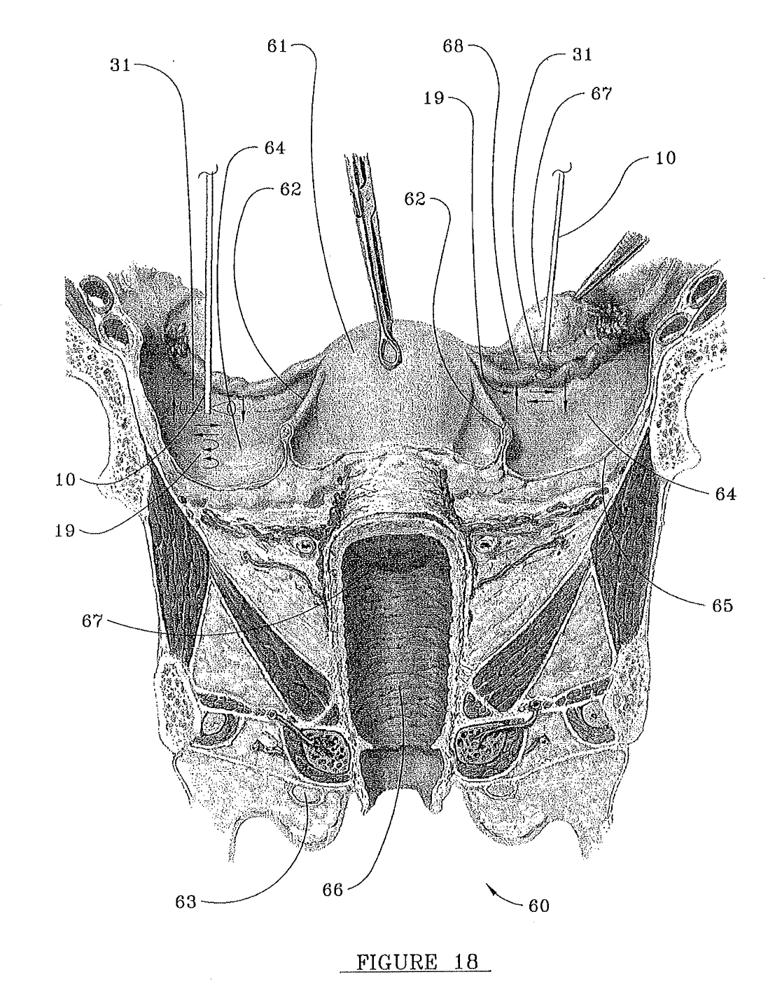

FIG. 18 illustrates the elements of the female reproductive System 60. Uterus 61 is held in place by round ligaments 62. The termination points 63 of round ligaments 62 are also shown. Uterus 61 is also held in place by broad ligaments 64, which terminate at peritoneum 65, defining the bottom of the abdominal cavity.

Vagina 66, cervix 67, fallopian tube 68 and ovary 69 are also shown.

Any of side firing devices 10 or FIG. 1-3, 5 or 7 may be inserted through a puncture in the abdomen, up to broad ligaments 64 or round ligaments 62, observed by a laparoscope inserted through a separate puncture in the abdomen, or inserted through the instrument channel of an endoscope, inserted through a puncture in the abdomen. Adjacent tissues may be moved away by one or more blunt or round-ended obturators, which are inserted through one or more separate punctures in the abdomen.

As shown, device 10 of FIG. 1-3, 5 or 7 may be Moved and advanced or withdrawn, Moved to the left or right and Rotated, concomitantly or in any desired sequence, to Sweep Holmium laser energy beam 31 (or other Source of Thermal Energy) over and shrink broad ligaments 64, as indicated by arrows 19. During lasing, a cooling fluid, such as sterile water or saline, should be infused through device 10, or through a separate cannula or needle, a laparoscope or an endoscope (none of which are separately shown).

Also as shown, any of devices 10(a-d) of FIG. 16 or 17, preferably those of FIG. 17, as they have a fluid channel to cool the Target Tissue, may be inserted, as described above, and device 10 and Holmium laser beam 31 (or other laser beam) may be Moved, as shown by arrows 19, to Sweep laser energy beam 31 along and Alter by shrinkage round ligaments 62.

If a cooling fluid is not infused through device 10, a cooling fluid may be infused through a laparoscope, endoscope, cannula or needle, as described above. In the absence of a cooling liquid to cool the Target Tissue, only low levels of laser energy should be used, for example, about 0.1 to 3 watts.

Shrinkage of both of round ligaments 62 and both of broad ligaments 64 lifts uterus 61 and Treats (reduces or eliminates) female stress urinary incontinence or “FSUI”.

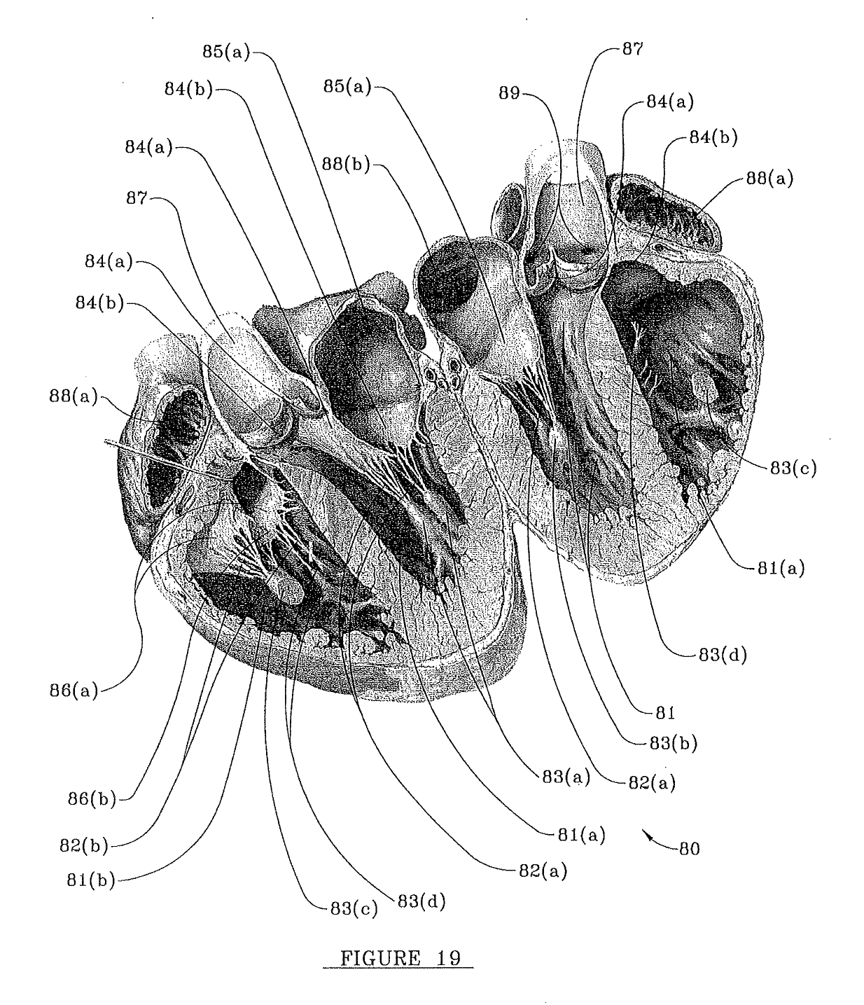

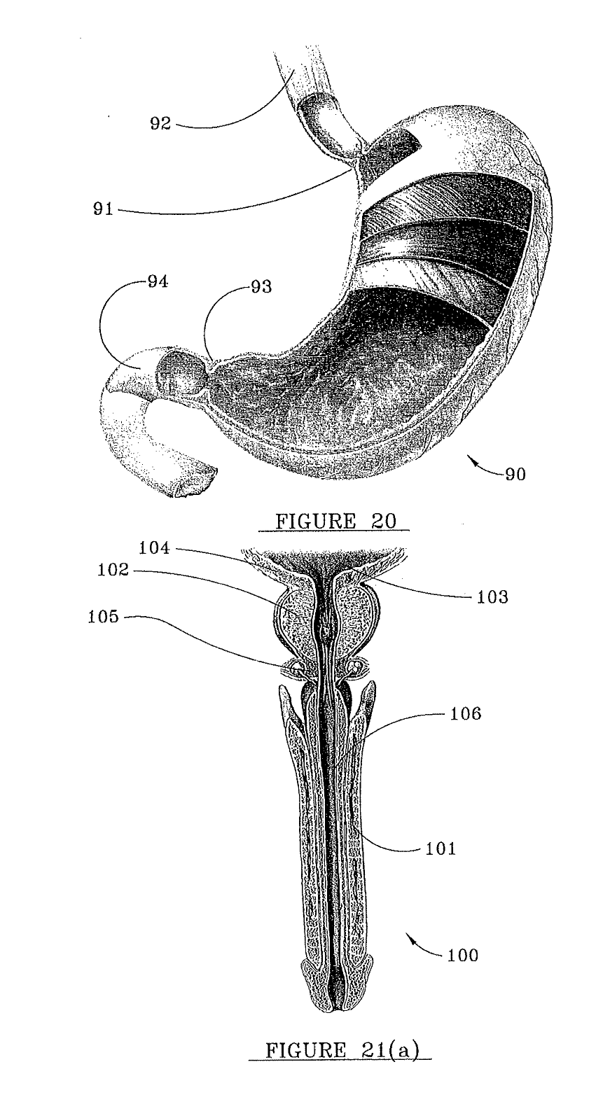

In FIG. 19, sections of heart 70 show the left ventricle 71(a) and right ventricle 71(b). Chordae tendinae 72(a) extend from anterior papillary muscle 73(a) and posterior papillary muscle 73(b) and terminate at anterior cusps 74(a) and posterior cusps 74(b) of the aortic valve, chordae tendinae 72(b) extend from anterior papillary muscle 73(c) and posterior papillary muscle 73(d) to anterior cusps 75(a) and posterior cusps 75(b) of the mitral valve, and chordae tendonae 72(c) extend from anterior papillary muscle 73(e) and posterior papillary muscle 73(e) to anterior cusps 86(a) and posterior cusps 86(b) of the tricuspid valve.

Also shown are aorta 87, right auricle 88(a) and left auricle 88(b), and the openings to the coronary arteries 89.

The optimal time to apply Holmium laser energy (or other Thermal Energy) to shrink the chordae tendinae 82(a)-(c) to Treat a prolapsed mitral, aortic or tricuspid heart valve, respectively, is during systole, when papillary muscles 83(a-d) are relaxed, releasing tension on chordae tendonae 82(a)-(c), respectively, as these tendons shrink to a greater degree when not under tension, as described heretofore.

However, if only about 10% shrinkage of chordae tendinae 82(a), (b) or (c) is desired, they may be shrunk during diastole, when papillary muscles 83(a-d) contract and joint chordae (a-c) under tension.

Preferably, this procedure is performed during bypass surgery or other open-heart procedure, before the heart is arrested, observed by color Doppler ultrasound imaging, as described heretofore. After shrinking chordae 82(a), (b) or (c), depending on which valve is prolapsed, after shrinking of the appropriate chordae 82(a), (b) or (c), if blood is still seen leaking from a prolapsed valve, laser energy emission port 18 of device 10 may be withdrawn to the top of the valve, called the annulus (not separately shown), to shrink the annulus and help stop the leaking.

FIG. 20 illustrates stomach 90, esophageal sphincter 91 of esophagus 92, pyloric valve sphincter 93 of pyloric valve 94 and duodenum 95. Any of side firing devices 10 of FIG. 1-5, 7, 9 or 11 may be disposed within a gastroscope (not separately shown) and advanced up to sphincter 91 of esophagus 92 and/or sphincter 93 of the pyloric valve 94. Preferably, device 10 of FIG. 9 or 11 should be used, as these devices have a round or concentrically shaped balloon 40, an eccentrically shaped balloon 40, or a back mounted balloon 40, to (a) center laser energy emission port 18 of device 10 in esophageal sphincter 91 and/or pyloric valve 94, to bring the laser energy emission port 18 of device 10 close to esophageal sphincter 91 and/or pyloric valve sphincter 93, or (c) press the energy emission port 18 of device 10 very close to esophageal sphincter 91, and/or pyloric valve sphincter 93, respectively.

As described above, balloon 40 may be inflated and device 10 may be positioned and aimed to emit Holmium laser energy (or any other Thermal Energy), for example, at 3 o'clock and, while laser energy is emitted, with concomitant infusion of a cooling fluid, as described above, device 10 is Rotated through an arc of about 90°, from about 1:30 to 4:30 o'clock and back, at the rate of about one arc per second. The balloon is deflated and device 10 is positioned and aimed to emit laser energy, for example, at 6 o'clock, the balloon is inflated and, while laser energy is emitted, with concomitant infusion of a cooling fluid, device 10 is Rotated through an arc of about 90°, from about 4:30 to 7:30 o'clock, and back. After which this balloon inflation, positioning, aiming, lasing and balloon deflation process is repeated with device 10 Stationed and aimed, successively, at 9 o'clock and then at 12 o'clock.

The above positionings and aimings can be made in any designed sequence to shrink sphincters 91 and/or 93. Also, instead of four cycles of 90° each, three cycles of 120° each, two cycles of 180° each, or any other number and length of cycles may be used.

If device 10 is Rotated through an arc greater than 90°, balloon 40 may be damaged. Alternatively, the proximal and distal ends of balloon 40 may be attached to a circular gasket (not separately shown), which maintains a water tight seal with and is moveably disposed between two ridges (not separately shown) on the exterior of device 10. This enables device 10 to be Rotated within balloon 40, through an arc greater than 90°, without damaging balloon 40, and without having to successively inflate and deflate balloon 40 in the positioning, aiming and lasing process.

Preferably, during the emission of laser energy, the infusion of an irrigation fluid, such as sterile water or saline, infused through the gastroscope, as well as through device 10 to flush debris from and cool the optical components of device 10 and cool the Target Tissue. This enables 3 to 40 watts of Holmium laser power, preferably about 5 to 20 watts, to be used in each of sphincter 91 and/or 93, whichever it is desired to Treat.

Tightening esophageal sphincter 91 reduces or prevents acidic liquids from stomach 80 to enter and erode esophagus 92 to Treat gastro-esophageal reflux disease or “GERD”.

Tightening sphincter 93 causes a reduction in the volume of food released from stomach 90 into duodenum 95. Retaining food in stomach 90 maintains the feeling of fullness or satiety, the patient ceases eating and weight is lost or, at least, weight gain is reduced or prevented.

Reducing weight (and exercising) has been shown to reduce or eliminate Type 2 diabetes, which affects millions of people throughout the world, causes a variety of adverse effects and is a major cost to the healthcare system, as described above.

If no cooling fluid is infused, much lower levels of laser energy for shorter periods of time is required to avoid damage to sphincters 91 and 93, as well as adjoining tissues, as described heretofore.

As described in co-owned U.S. Pat. No. 6,635,052, which is fully incorporated herein by reference, one or more needles, with sharp or syringe-like distal ends (not separately shown), composed of a resilient material, such as a memory metal or Nitinol, which, when straightened during passage through the instrument channel of an endoscope or a lumen of a rigid, semi-rigid or flexible cannula 15, resumes their initial bent shape, for example, of about 70° to 90°. Each needle contains an optical fiber and may be inserted into esophageal sphincter 91 and/or pyloric valve sphincter 93 to Alter by shrinkage sphincters 91 and/or 93.

Whereas, in the present invention, shrinkage of a Target Tissue to Treat a Medical Condition is accomplished externally, without insertion of devices 10 of FIG. 1-5, 7, 9 or 11 into sphincters 91 and/or 93.

As described in co-owned U.S. Pat. No. 6,740,107, which is fully incorporated herein by reference, the distal end portion of a side firing optical fiber device is contained within an eccentrically shaped balloon, which is inserted into the femoral artery in the groin and is moved through a conventional guiding catheter into the left ventricle to treat mitral valve prolapse. The eccentricity of the balloon, when inflated by the infusion of a radioopaque fluid, enables the cardiologist by x-ray imaging to determine the direction of laser energy emission.

Whereas, in the present invention, devices 10 of FIG. 1-5, 7, 9 or 11 are designed for Treating heart valve prolapse during open heart surgery or bypass surgery, and are not designed for insertion into the femoral artery in the groin and being advanced through a guiding catheter into a ventricle of the heart to treat valve prolapse in a percutaneous procedure under x-ray imaging, which requires a device 10 with a much longer working length than 80 cm.

FIG. 21(a) illustrates the male penis 100, urethra 101 and urethral sphincters 102. Any of rigid side firing devices 10 of FIG. 1-5, 7, 9 or 11 may be disposed in a rigid endoscope which is lubricated and inserted into urethra 101 of penis 100. Device 10 is extended from the instrument channel of the endoscope and is Stationed opposite urethral sphincter 102. While Holmium laser energy (or other Source of Thermal Energy) is emitted, device 10 is Rotated, as described above, while a cooling fluid of sterile saline or water is infused through the endoscope, as well as from device 10 to clean and cool the optical components of device 10 and to cool the Target Tissue. If no cooling fluid is infused, an extremely low level of laser energy must be used, as described above.

The Alteration of urethral sphincter 102 by shrinkage is performed to Treat male urinary incontinence.

FIG. 21(b) illustrates the female urinary system 110. Bladder 111, urethra 112 and sphincters 113 are shown. To Treat female urinary incontinence, the same procedure described with respect to FIG. 21(a) is performed to Alter by shrinkage urethra 102, as described above.

FIG. 22, illustrates the male or female rectal system 120. Any of rigid, side firing devices 10 of FIG. 1-5, 7, 9 or 11 may be disposed in the instrument channel of a rigid endoscope (not separately shown), which is lubricated and inserted into anus 121 and is advanced up to rectal sphincter 122. Side firing device 10 is extended from the endoscope and positioned opposite rectal sphincter 122. While Holmium laser energy (or other Thermal Energy) is emitted, accompanied by infusion of sterile water or saline through the endoscope, as well as device 10, for the reasons set forth above, device 10 is Rotated to Alter by shrinkage rectal sphincter 122 at one or more o'clock positions, as described above.

Then, the endoscope is withdrawn to just proximal to anal sphincter 123, and the above described procedure is repeated. Of course, anal sphincter 123 may be shrunk before rectal sphincter 122 is shrunk, if so desired by the surgeon.

As seen in FIG. 22, for example, side firing device 10 of FIG. 9 or 11, with a concentric, inflated balloon, centers side firing device 10 in anal sphincter 123. While Holmium laser energy (or other Thermal Energy) is emitted, side firing device 10 is Rotated, as described above, and cooling fluid, such as sterile water or saline, is infused through endoscope, as well as through device 10, for the reasons set forth above, to Alter by shrinkage sphincters 122 and 123 to Treat male or female fecal incontinence. Of course, anal sphincter 123 can be shrunk before rectal sphincter 122.

While this invention is susceptible of embodiment in many different forms, these are shown in the drawings and will be described in detail herein specific embodiments thereof, with the understanding that the present disclosure is to be considered as an exemplification of the principles of the invention and is not to be limited to the specific embodiment illustrated.

Numerous variations and modifications of the embodiments described above can be effected without departing from the spirit and scope of the novel features of the invention. It is to be understood that no limitation with respect to the specific apparatus illustrated herein is intended or should be inferred. It is, of course, intended to cover by the appended claims, all such modifications as fall within the scope of the claims.

I claim:

1. A side firing optical fiber device which comprises an optical fiber for transmission of laser energy from a source of laser energy, and a metal end piece mounted to the fiber and defining a cavity with an open side port and receiving within the cavity a bared distal end portion of the optical fiber, the metal end piece further defining an inclined surface in the cavity at distal end portion thereof; the bared distal end portion being bonded to the metal end piece; the inclined surface being reflective of the laser energy, being disposed in the cavity opposite the distal end face of the optical fiber and capable of emitting laser energy at a wavelength in the range of 300 to 3000 nm and at an angle of about 90° laterally from the axis of the optical fiber.

2. The side firing optical fiber device of claim 1, wherein the metal end piece is made entirely of a metal selected from a group consisting of: gold and silver having a purity of at least about 90%.

3. The side firing optical fiber device of claim 1 wherein the purity of gold and silver is at least about 95.5%.

4. The side firing optical fiber device of claim 1 wherein core of the optical fiber has a hydroxyl ion content of no more than about 100 parts per million.

5. The side firing optical fiber device of claim 1 wherein core of the optical fiber has a hydroxyl ion content in the range of about 0.1 to 100 parts per million.

6. The side firing optical fiber device of claim 1 wherein core of the optical fiber has a hydroxyl ion content in the range of about 0.01 to 1 part per million.

7. The side firing optical fiber device of claim 1 wherein the optical fiber has an outside diameter of no more than 1.5 millimeters.

8. The side firing optical fiber device of claim 1 wherein the optical fiber has a core diameter less than 500 microns.

9. The side firing optical fiber device of claim 1 wherein the optical fiber has a core diameter of about 365 microns.

10. A side firing optical fiber device which comprises an optical fiber for transmission of laser energy from a source of laser energy, and a closed-end capillary tube sealingly encasing a distal end portion of the optical fiber; the optical fiber having a distal end face beveled at an angle of 35° to 45° and emitting laser energy at an angle of about 80° to 82° laterally from longitudinal axis of the optical fiber when coupled to the source of laser energy.

11. The optical fiber device of claim 10, wherein the distal end of the optical fiber is beveled providing two, chisel-like adjacent surfaces, each beveled at an angle of 40° to 41°, and each surface emitting laser energy at an angle of 80° to 82° from the axis of the optical fiber simultaneously when coupled to a source of laser energy.

12. The optical fiber device of claim 10, wherein the capillary tube has a wall thickness not greater than 350 microns.

13. The optical device of claim 10, wherein the optical fiber has a core diameter of not more than 365 microns and is bendable at an angle of up to 90° with a bend radius not less than 1.5 cm at a temperature of about 2° C. and a bend radius not less than 1 cm, at a temperature of about 0° C.

14. The optical fiber device of claim 10, provided with a double-walled, multi-channel plastic tube attached to the optical fiber for delivery of a sterile, biocompatible fluid at a rate sufficient to at least one of: (a) cool and flush debris from optical components in the tip of the side firing device and cool the Target Tissue; (b) inflate a round or concentric balloon to center the side firing device opposite a Target Tissue; (c) inflate an eccentric balloon to press the laser energy emitting surface of the side firing device close to the Target Tissue; (d) inflate a back-mounted balloon to press the laser energy emitting surface of the side firing device against the Target Tissue; (e) enable excess fluid to flow from the balloon to one of: a drain and a collection bottle; and (f) enable the balloon to be deflated by suction or by withdrawing a plunger of a syringe used to inflate the balloon.

15. The optical fiber device of claim 10, further provided with a balloon around the plastic tube at a distal end portion thereof and wherein the balloon defines at least one vent for removing excess fluid from the balloon.

16. The optical fiber device of claim 10 wherein a plastic tube is provided over the distal end portion of the optical fiber and the proximal end portion of the capillary tube.

17. The optical fiber device of claim 16 wherein the plastic tube is secured to the distal end portion of the optical fiber by an adhesive.

18. The optical fiber device of claim 17 wherein the adhesive is substantially transparent to laser energy and does not absorb more than 6 percent of laser energy passing through.

19. The optical fiber device of claim 10 wherein the optical fiber is situated in a cannula and the optical fiber together with the cannula define a fluid passageway therebetween.

20. The optical fiber device of claim 19 wherein the cannula is flexible.

21. The optical fiber device of claim 19 wherein the cannula is rigid.

22. The optical fiber device of claim 10 wherein the optical fiber is situated within a double walled, hollow tube which defines at least one fluid passageway along the optical fiber.

23. The optical fiber device of claim 22 wherein an expandable balloon eccentrically encases said double walled, hollow tube at a distal end portion of the tube and is in fluid communication with one said fluid passageway.

24. An apparatus for delivery of laser energy to a Target Tissue comprised of an optical fiber within a flexible metal cannula and defining a fluid passageway therebetween, the cannula being bendable up to about 60° from normal longitudinal axis and having at the proximal end portion thereof a coupling for delivery of a sterile, biocompatible fluid through the fluid passageway.

25. A method for Treating a Medical Condition of a Patient comprised of at least one of: positioning, Moving, Rotating and Sweeping Onto a Target Tissue laser energy which is one of: pulsed laser energy and continuous wave laser energy; the laser energy being delivered at a desired angle up to about 90° from the longitudinal axis of an optical fiber delivering the laser energy.

26. The method of claim 25, wherein the laser energy is delivered to a Target Tissue by one of: (a) multiple beams of laser energy focused to intersect at a desired point and (b) a beam of laser energy focused to converge at a desired point, the point being about 2 mm to 5 mm from the laser energy emitting surface of the device, to shrink a Target Tissue.

27. The method of claim 25, wherein the laser energy is a CTH:YAG laser, which shrinks sphincters with less thermal damage to tissue, as it allows significant time between pulses of laser energy for the tissue to cool.

28. A method for Treating a Medical Condition of a Patient comprised of at least one of: Stationing, Moving, Rotating and Sweeping Onto a Target Tissue thermal energy to Alter by shrinkage at least one of: the esophageal sphincter, the sphincter of the pyloric valve of the stomach and the sphincter of at least one of: (a) the urethra, which has a moderate collagen content, (b) the anus, which has a moderate collagen content, and (c) the round and broad ligaments of the uterus, which have a high collagen content, the Medical Condition of the Patent being at least one of: gastroesophageal reflux disease (GERD), obesity, Type 2 diabetes, urinary incontinence, fecal incontinence, female stress urinary incontinence and other sphincter-related Medical Conditions.

29. The method of claim 28, wherein the thermal energy is a CTH:YAG laser, which shrinks sphincters with less thermal damage to tissue, as it allows significant time between pulses of laser energy for the tissue to cool.

30. The method of claim 28, wherein the thermal energy is delivered to a Target Tissue by a device which emits one of: (a) multiple beams of laser energy focused to intersect at a point and (b) a beam of laser energy focused to converge at a point, the point being about 2 mm to 5 mm from the laser energy emitting surface of the device.
